(19) United States
(12) Patent Application Publication
(10) Pub. No.: US 2004/0023248 A1
(43) Pub. Date: Feb. 5, 2004

(54) Methods and reagents for improving nucleic acid detection

(75) Inventor: Shawn M. O'Malley, Millville, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institiute for Biomedical Research, Cambridge, MA

(21) Appl. No.: 10/314,901

(22) Filed: Dec. 9, 2002

Related U.S. Application Data
(60) Provisional application No. 60/341,094, filed on Dec. 7, 2001.

(51) Int. Cl. C12Q 1/68; C08G 63/48; C08G 63/91

(52) U.S. Cl. 435/6; 525/54.2

(57) ABSTRACT

The present invention features methods for enhancing nucleic acid molecule detection through the use of dendrimers. Novel dendrimers and methods of making such dendrimers are also features of the present invention.

### RELATED APPLICATION

This application claims the benefit of U.S. Provisional Application No. 60/341,094, filed Dec. 7, 2001. The entire teachings of the above application are incorporated herein by reference.

### BACKGROUND OF THE INVENTION

High density nucleic acid microarrays are used for the simultaneous and rapid detection of many expressed genes through hybridization techniques. These techniques are also known as high density display. Entire populations of mRNAs from a given sample can be hybridized to the high density nucleic acid microarrays in order to detect and measure gene expression. High density display is useful for detecting, diagnosing and predicting diseases and determining the levels and timing of gene expression.

High density display is most useful when it is sensitive, reliable, and accurate. The manner in which nucleic acid molecules are labeled for detection using these microarray methods affects the ability of the nucleic acid to be detected sensitively, reliably, and accurately. The nucleic acid molecules to be detected (target nucleic acid molecules) are generally in the form of cRNAs which have been generated using reverse transcription and in vitro transcription techniques. Often fluorescently modified molecules (e.g., fluorescent dNTPs) are directly incorporated into the reverse transcription reaction during preparation of the target nucleic acid molecules that are to be detected. The amount of fluorescent dNTPs incorporated into each target nucleic acid molecule is sequence dependent, and therefore it is not possible to reliably quantify target nucleic acid molecules bound to a microarray based on the fluorescence intensity of the signal without performing a relative ratio comparison control. In addition, high levels of dye incorporation into target nucleic acid molecules can interfere with subsequent detection steps.

Alternative methods that increase the sensitivity of detection systems and involve a minimal number of steps for microarray detection would provide for improved analyses of gene expression products and disease detection systems.

### SUMMARY OF THE INVENTION

The present invention features methods for improving detection of nucleic acid molecules using microarrays, and compounds for use in such detection methods. The detection methods involve the use of dendrimers that are detectably labeled. Dendrimers are spherical polymers or biopolymers of defined molecular mass and structure, and are formed in a step-wise series of layers (generations) (Hudson and Damha, J. Am. Chem. Soc. 115:2119-2124, 1993; and Esfand and Tomalia, Drug Discov. Today 6:427-436, 2001). The detectably labeled dendrimers of the present invention recognize and bind to a target nucleic acid molecule in a number of different methods. The target nucleic acid molecule dendrimer complex is then detected via the detectable label of the dendrimer. In addition, novel dendrimers for use in the target nucleic acid detection methods are also a feature of the present invention.

Accordingly, in one aspect, the invention features a dendrimer comprising a nucleic acid molecule conjugated to a dendrimeric polymer, wherein the dendrimeric polymer is not a DNA dendrimeric polymer. In one embodiment, the dendrimeric polymer is a poly(amidoamine) polymer. In other embodiments, the dendrimer polymer is a cyclotriphosphazene-phenoxymethyl(methylhydrazono) dendrimer, a polypropylenimine dotriacontaamine dendrimer, or a thiophosphoryl-phenoxymethyl(methylhydrazono) dendrimer. In another embodiment, the nucleic acid molecule is an oligonucleotide or a peptide nucleic acid molecule. In another embodiment, the dendrimeric polymer comprises a generation zero (0) dendrimer (which has 4 functional groups). In still another embodiment, the dendrimeric polymer comprises a generation one or higher polymer, for example, a generation two, three, four, five, six or seven dendrimeric polymer, and the nucleic acid molecule is conjugated to the highest generation of the polymer. In yet another embodiment, the dendrimer is detectably labeled. Preferably, the detectably labeled dendrimer is labeled with an agent selected from the group consisting of a hapten, for example, biotin, a fluorescent label, an enzyme, and a radioactive isotope. For example, the agent can be a fluorescent label selected from the group consisting of CY3, CY5, ALEXA FLUOR 594, FAM, ROX, TAMRA, ALEXA 488, JOE, and fluorescein. In still another embodiment, the dendrimeric polymer comprises a generation zero or higher polymer, for example, a generation one, two, three, four, five, six, or seven dendrimeric polymer, and the dendrimer is detectably labeled on the highest generation of the polymer.

In another aspect, the invention features a method of detecting a target nucleic acid molecule, comprising the steps of: a) contacting a target nucleic acid molecule with a bipartite capture probe comprising a target nucleic acid molecule capture region and a dendrimer capture region, under conditions that allow hybridization of the bipartite capture probe to the target nucleic acid molecule, thereby forming a target nucleic acid molecule bound capture probe; b) contacting the target nucleic acid molecule bound capture probe with a detectably labeled dendrimer, said dendrimer comprising a dendrimeric polymer having one or more single stranded capture probe nucleic acid binding sequences attached to it, under conditions that allow hybridization of the dendrimer capture region of the bipartite capture probe to the single stranded capture probe binding sequence, thereby forming a labeled target nucleic acid molecule bound capture probe-dendrimer complex; and c) detecting the labeled target nucleic acid molecule bound capture probe-dendrimer complex, thereby detecting the target nucleic acid molecule.

In one embodiment of the above aspect of the invention, the target nucleic acid molecule is immobilized on a solid support, for example, a microarray. In another embodiment, the bipartite capture probe is from about 60 nucleotides to about 90 nucleotides in length. In another embodiment, the detectably labeled dendrimer is labeled with an agent selected from the group consisting of a hapten, for example, biotin, a fluorescent label, an enzyme, and a radioactive isotope. For example, the fluorescent label can be selected from the group consisting of CY3, CY5, ALEXA FLUOR 594, FAM, ROX, TAMRA, ALEXA 488, JOE, and fluorescein. In another embodiment, the detectably labeled DNA dendrimer comprises from 1 to about 2048 detectable labels, for example, about 4, 8, 32, 128, or 512 detectable labels. In one embodiment, the label is biotin. In other embodiments, the bipartite capture probe is a linear molecule or a branched molecule. In still another embodiment, the target nucleic acid molecule is a single stranded nucleic acid molecule. In yet other embodiments, the dendrimer is a poly(amidoamine) dendrimer, a cyclotriphosphazene-phenoxymethyl(methylhydrazono) dendrimer, a polypropylenimine dotriacontaamine dendrimer, a thiophosphoryl-phenoxymethyl(methylhydrazono) dendrimer, or a DNA dendrimer. In another embodiment, the target nucleic acid molecule is specific for a cell phenotype, and by detecting the target nucleic acid molecule, the cell phenotype is detected.

In another aspect, the invention features a method of detecting a target nucleic acid molecule, comprising the steps of: a) contacting a biotin-labeled target nucleic acid molecule with streptavidin under conditions that allow binding of the streptavidin to the biotin-labeled target nucleic acid molecule, thereby forming a biotin-labeled target nucleic acid molecule-streptavidin complex; b) contacting the biotin-labeled target nucleic acid molecule-streptavidin complex with a biotin-labeled nucleic acid capture probe comprising a dendrimer capture region, under conditions that allow binding of the biotin-labeled capture probe to the biotin-labeled target nucleic acid molecule-streptavidin complex, thereby forming a biotin-labeled target nucleic acid bound capture probe; c) contacting the biotin-labeled target nucleic acid bound capture probe with a detectably labeled dendrimer, said dendrimer comprising one or more single stranded capture probe nucleic acid binding sequences attached to it, under conditions that allow hybridization of the dendrimer capture region of the biotin-labeled target nucleic acid bound capture probe to the single stranded capture probe nucleic acid binding sequence, thereby forming a biotin-labeled target nucleic acid bound capture probe-detectably labeled dendrimer complex; and d) detecting the biotin-labeled target nucleic acid bound capture probe-detectably labeled dendrimer complex, thereby detecting the target nucleic acid molecule.

In one embodiment of the above aspect of the invention, the target nucleic acid molecule is immobilized on a solid support, for example, a microarray. In another embodiment, the capture probe comprises nucleic acids and is from about 18 nucleotides to about 30 nucleotides in length. When the capture probe is comprised of peptide nucleic acids (PNAs) the capture probe is preferably from about 12 peptide nucleic acids to about 18 peptide nucleic acids in length. In another embodiment, the detectably labeled DNA dendrimer comprises from 1 to about 2048 detectable labels, for example, about 4, 8, 32, 128, or 512 detectable labels. In one embodiment, the detectable label is biotin. In addition, the detectably labeled dendrimer can be labeled internally, for example, by internally labeling the DNA or other recognizing hybridizing element (e.g., PNA) that binds the target nucleic acid, such that the total number of labels is increased. In another embodiment, the detectably labeled dendrimer further comprises a detectable label that is not biotin and wherein in step d) detection of the biotin-labeled target nucleic acid bound capture probe-detectably labeled dendrimer complex occurs by detecting the detectable label. The detectable label can be an agent selected from the group consisting of a hapten, a fluorescent label, an enzyme, and a radioactive isotope. For example, the fluorescent label can be selected from the group consisting of CY3, CY5, ALEXA FLUOR 594, FAM, ROX, TAMRA, ALEXA 488, JOE, and fluorescein. In other embodiments, the capture probe is a linear molecule or a branched molecule. In still another embodiment, the target nucleic acid molecule is a single stranded nucleic acid molecule. In yet other embodiments, the dendrimer is a poly(amidoamine) dendrimer, a cyclotriphosphazene-phenoxymethyl(methylhydrazono) dendrimer, a polypropylenimine dotriacontaamine dendrimer, a thiophosphoryl-phenoxymethyl(methylhydrazono) dendrimer, or a DNA dendrimer. In another embodiment, the target nucleic acid molecule is specific for a cell phenotype, and by detecting the target nucleic acid molecule, the cell phenotype is detected.

In another aspect, the invention features a method of detecting a target nucleic acid molecule by immunodendrimer capture, comprising the steps of: a) contacting a hapten-labeled target nucleic acid molecule with a capture probe comprising a dendrimer capture region, wherein the capture probe is conjugated to an antibody that binds the hapten, under conditions that allow binding of the capture probe to the hapten-labeled target nucleic acid molecule, thereby forming a target nucleic acid bound capture probe; b) contacting the hapten-labeled target nucleic acid bound capture probe with a detectably labeled dendrimer, said dendrimer comprising one or more single stranded capture probe nucleic acid binding sequences attached to it, under conditions that allow hybridization of the dendrimer capture region of the hapten-labeled target nucleic acid bound capture probe to the single stranded capture probe nucleic acid binding sequence, thereby forming a hapten-labeled target nucleic acid bound capture probe-detectably labeled dendrimer complex; and c) detecting the hapten-labeled target nucleic acid bound capture probe-detectably labeled dendrimer complex, thereby detecting the target nucleic acid molecule.

In one embodiment of the above aspect of the invention, the hapten is biotin. In another embodiment, the target nucleic acid molecule is immobilized on a solid support, for example, a microarray. In another embodiment, the capture probe comprises nucleic acids and is from about 18 nucleotides to about 30 nucleotides in length. When the capture probe is comprised of PNAs, the capture probe is preferably from about 12 peptide nucleic acids to about 18 peptide nucleic acids. In another embodiment, the detectably labeled DNA dendrimer comprises from 1 to about 2048 detectable labels, for example, about 4, 8, 32, 128, 512 detectable labels, for example, biotin. In addition, the detectably labeled dendrimer can be labeled internally such that the total number of detectable labels is increased; for example, DNAs or other hybridizing elements (e.g., PNAs) of detectably labeled dendrimers can be internally labeled). In another embodiment, when the hapten is biotin, the detectably labeled dendrimer further comprises a detectable label that is not biotin, and wherein in step c) detecting the hapten-(biotin) labeled target nucleic acid bound capture probe-detectably labeled dendrimer complex occurs by detecting the detectable label. Preferably, the detectable label is an agent selected from the group consisting of a hapten other than biotin, a fluorescent label, an enzyme, and a radioactive isotope. For example, the fluorescent molecule can be selected from the group consisting of CY3, CY5, ALEXA FLUOR 594, FAM, ROX, TAMRA, ALEXA 488, JOE, and fluorescein. In other embodiments, the capture probe is a linear molecule or a branched molecule. In still another embodiment, the target nucleic acid molecule is a single stranded nucleic acid molecule. In yet other embodiments, the dendrimer is a poly(amidoamine) dendrimer, a cyclotriphosphazene-phenoxymethyl(methylhydrazono) dendrimer, a polypropylenimine dotriacontaamine dendrimer, a thiophosphoryl-phenoxymethyl(methylhydrazono) dendrimer, or a DNA dendrimer. In another embodiment, the target nucleic acid molecule is specific for a cell phenotype, and by detecting the target nucleic acid molecule, the cell phenotype is detected.

In another aspect, the invention features a method of detecting a target nucleic acid molecule, comprising the steps of: a) contacting a biotin-labeled target nucleic acid molecule and a biotin-labeled dendrimer with streptavidin under conditions that allow binding of the biotin-labeled target nucleic acid molecule and the biotin-labeled dendrimer to the same streptavidin molecule, thereby forming a biotin-labeled target nucleic acid molecule-dendrimer complex; and b) detecting the biotin-labeled target nucleic acid molecule-dendrimer complex, thereby detecting the target nucleic acid molecule.

In one embodiment of the above aspect of the invention, the target nucleic acid molecule is immobilized on a solid support, for example, a microarray. In another embodiment, in step b) detection of the biotin-labeled target nucleic acid molecule-detectably labeled dendrimer complex occurs by detecting biotin. In another embodiment, the biotin-labeled dendrimer comprises from 1 to about 2048, for example, about 4, 8, 32, 128, or 512, biotin molecules. In addition, the detectably labeled dendrimer can be labeled internally such that the total number of labels is increased; for example, DNAs or other hybridizing elements (e.g., PNAs) of detectably labeled dendrimers can be internally labeled. In still another embodiment, the detectably labeled dendrimer further comprises a detectable label that is not biotin, and wherein in step b) detecting the biotin-labeled target nucleic acid molecule-detectably labeled dendrimer complex occurs by detecting the detectable label. Preferably, the detectable label is an agent selected from the group consisting of a hapten other than biotin, a fluorescent label, an enzyme, and a radioactive isotope. For example, the fluorescent label can be selected from the group consisting of CY3, CY5, ALEXA FLUOR 594, FAM, ROX, TAMRA, ALEXA 488, JOE, and fluorescein. In still another embodiment, the detectably labeled dendrimer comprises from 1 to about 2048, for example, about 4, 8, 32, 128, or 512, detectable labels. In still another embodiment, the target nucleic acid molecule is a single stranded nucleic acid molecule. In yet other embodiments, the dendrimer is a poly(amidoamine) dendrimer, a cyclotriphosphazene-phenoxymethyl(methylhydrazono) dendrimer, a polypropylenimine dotriacontaamine dendrimer, a thiophosphoryl-phenoxymethyl(methylhydrazono) dendrimer, or a DNA dendrimer. In another embodiment, the target nucleic acid molecule is specific for a cell phenotype, and by detecting the target nucleic acid molecule, the cell phenotype is detected.

In another aspect, the invention features a method of detecting a target nucleic acid molecule, comprising the steps of: a) contacting a biotin-labeled target nucleic acid molecule with streptavidin under conditions that allow binding of the streptavidin to the biotin-labeled target nucleic acid molecule, thereby forming a target nucleic acid molecule-streptavidin complex; b) contacting the target nucleic acid molecule-streptavidin complex with a biotin-labeled nucleic acid capture probe comprising a first dendrimer capture region, under conditions that allow binding of the biotin-labeled nucleic acid capture probe to the target nucleic acid molecule-streptavidin complex, thereby forming a target nucleic acid molecule bound capture probe; c) contacting the target nucleic acid molecule bound capture probe with a first detectably labeled dendrimer, said first dendrimer comprising one or more single stranded capture probe nucleic acid binding sequences and one or more single stranded second biotin-labeled dendrimer capture nucleic acid sequences, under conditions that allow hybridization of the first dendrimer capture region of the target nucleic acid bound capture probe to the single stranded capture probe nucleic acid binding sequence, thereby forming a target nucleic acid molecule bound capture probe-first dendrimer complex; d) contacting the target nucleic acid molecule bound capture probe-first dendrimer complex with a second detectably labeled dendrimer comprising one or more nucleic acid sequences that hybridize to the first dendrimer, under conditions that allow hybridization of the target nucleic acid bound capture probe-first dendrimer complex to the second detectably labeled dendrimer, thereby forming a target nucleic acid molecule bound capture probe-nested detectably labeled dendrimer complex; and e) detecting the target nucleic acid molecule bound capture probe-nested detectably labeled dendrimer complex, thereby detecting said target nucleic acid molecule.

In one embodiment of the above aspect of the invention, the target nucleic acid molecule is immobilized on a solid support, for example, a microarray. In another embodiment, each first and second detectably labeled dendrimer comprise from 1 to about 2048, for example, about 4, 8, 32, 128, or 512 detectable labels, for example, biotin. The detectable labels of the first dendrimer and the detectable labels of the second dendrimer can be different, but preferably are the same. In addition, DNAs or other recognizing hybridizing elements (e.g., PNAs) of the detectably labeled dendrimers can be labeled internally such that the total number of labels is increased. In yet another embodiment, the first labeled dendrimer and the second labeled dendrimer further comprise a detectable label that is not biotin, and wherein in step e) detection of the target nucleic acid molecule bound capture probe-nested detectably labeled dendrimer complex occurs by detecting the detectable label. Preferably, the detectable label is an agent selected from the group consisting of a hapten, a fluorescent molecule, an enzyme, and a radioactive isotope. More preferably, the agent is a fluorescent molecule selected from the group consisting of CY3, CY5, ALEXA FLUOR 594, FAM, ROX, TAMRA, ALEXA 488, JOE, and fluorescein. In still another embodiment, the detectably labeled dendrimer comprises from 1 to about 2048, for example, about 4, 8, 32, 128, or 512, detectable labels. In still another embodiment, the target nucleic acid molecule is a single stranded nucleic acid molecule. In yet other embodiments, each of the first and second dendrimers is a poly(amidoamine) dendrimer, a cyclotriphosphazene-phenoxymethyl(methylhydrazono) dendrimer, a polypropylenimine dotriacontaamine dendrimer, a thiophosphoryl-phenoxymethyl(methylhydrazono) dendrimer, or a DNA dendrimer. In another embodiment, the target nucleic acid molecules is specific for a cell phenotype, and by detecting the target nucleic acid molecule, the cell phenotype is detected.

In still another embodiment, the invention features a method of detecting a target nucleic acid molecule, comprising the steps of: a) contacting a hapten labeled target nucleic acid with a capture probe comprising a dendrimer capture region, the capture probe conjugated to an antibody that binds the hapten, under conditions that allow binding of the capture probe to the hapten-labeled target nucleic acid molecule, thereby forming a target nucleic acid bound capture probe; b) contacting the hapten-labeled target nucleic acid bound capture probe with a dendrimer, the dendrimer comprising one or more nucleic acid sequences, the nucleic acid sequences further comprising single stranded capture probe nucleic acid binding sequences attached to it, under conditions that allow hybridization of the dendrimer capture region of the hapten-labeled target nucleic acid bound capture probe to the single stranded capture probe nucleic acid binding sequence, thereby forming a hapten-labeled target nucleic acid bound capture probe-dendrimer complex; and c) detecting the hapten-labeled target nucleic acid bound capture probe-dendrimer complex by amplifying a nucleic acid sequence on the dendrimer, and detecting said amplification products, thereby detecting the target nucleic acid molecule.

In one embodiment of the above aspect of the invention, the hapten is biotin. In another embodiment, the target nucleic acid molecule is immobilized on a solid support, for example, a microarray. In another embodiment, the capture probe comprises nucleic acids and is from about 18 nucleotides to about 30 nucleotides in length. When the capture probe is comprised of PNAs, the capture probe is preferably from about 12 peptide nucleic acids to about 18 peptide nucleic acids. In another embodiment, the detectably labeled DNA dendrimer comprises from 1 to about 2048 detectable labels, for example, about 4, 8, 32, 128, 512 detectable labels, for example, biotin. dendrimer complex occurs by detecting the detectable label. In other embodiments, the capture probe is a linear molecule or a branched molecule. In still another embodiment, the target nucleic acid molecule is a single stranded nucleic acid molecule. In yet other embodiments, the dendrimer is a poly(amidoamine) dendrimer, a cyclotriphosphazene-phenoxymethyl(methylhydrazono) dendrimer, a polypropylenimine dotriacontaamine dendrimer, a thiophosphoryl-phenoxymethyl(methylhydrazono) dendrimer, or a DNA dendrimer. In another embodiment, the target nucleic acid molecule is specific for a cell phenotype, and by detecting the target nucleic acid molecule, the cell phenotype is detected. In still another embodiment, in step c) amplification of the nucleic acid sequences in the dendrimer is done using the polymerase chain reaction or in vitro transcription.

### BRIEF DESCRIPTION OF THE DRAWINGS

FIG. 1 is a schematic representation of the steps involved in using bipartite capture probes to detect target nucleic acid molecules.

FIG. 2 is a schematic representation of steps involved in using dendrimer encapsulated polymers as catalysts for nucleic acid molecule detection.

FIG. 3 is a graph of the effect of DNA on COOH PAMAM dendrimers and NH2 PAMAM dendrimers.

FIG. 4 is a scanned image of a 1.8% agarose gel (TBE) stained with GELSTAR nucleic acid stain. Lane 1 shows a molecular weight marker; Lane 2 shows unlabeled 7.5 kDa oligonucleotide CP3; Lane 3 shows unlabeled 10.3 kDa oligonucleotide CP4; Lane 4 shows 12.7 kDa oligonucleotide Cy3 plus dendrimer without conjugation; Lane 5 shows 12.7 kDa oligonucleotide Cy5 plus dendrimer without conjugation; Lane 6 shows 0.2 μL of Cy5 DNA-dendrimer conjugate; Lane 7 shows 6 μL of Cy3 DNA-dendrimer conjugate; Lane 8 shows 2 μL of Cy3 DNA-dendrimer conjugate; Lane 9 shows 6 μL of Cy5 DNA-dendrimer conjugate; and Lane 10 shows 2 μL of Cy5 DNA-dendrimer conjugate.

FIG. 5A is a scanned image of a slide of PCR amplicons for the IL1RN gene stained with an oligonucleotide stain having just one Cy5 dye.

FIG. 5B is a scanned image of a slide of PCR amplicons for the IL1RN gene stained with Cy5 labeled DNA-dendrimers.

FIG. 6A is a scanned image of a biotin spotted array in which streptavidin was used to block all of the spotted biotin on the array prior to detection using a standard streptavidin phycoerythrin staining protocol.

FIG. 6B is a scanned image of a biotin spotted array detected using a standard streptavidin phycoerythrin staining protocol.

FIG. 6C is a scanned image of a biotin spotted array detected using a biotin labeled COOH-PAMAM dendrimer.

FIG. 6D is a scanned image of a biotin spotted array detected using a biotin labeled NH₂-PAMAM dendrimer.

FIG. 6E is a histogram of a comparison of streptavidin phycoerythrin intensity to biotin dendrimer stain intensity on the spotted biotin arrays described in FIGS. 6A-6D (“stv-sape” is the spotted biotin array described in FIG. 6A; “sape” is the spotted biotin array described in FIG. 6B; “biotin COOH” is the spotted biotin array described in FIG. 6C; and “biotin HN₂” is the spotted biotin array described in FIG. 6D).

FIG. 7A is a scanned image of a U95B Affymetrix chip hybridized with 1 μg of cRNA and stained with a streptavidin phycoerythrin stain using a standard method.

FIG. 7B is a scanned image of a U95B Affymetrix chip hybridized with 1 μg of cRNA and stained using a biotin COOH PAMAM dendrimer stain.

FIG. 8A is a graph of a comparison of the scanned images shown in FIGS. 7A and 7B, indicating the correlation between the detection methods described in FIGS. 7A and 7B.

FIG. 8B is a histogram of the distribution of the fold signal amplification yielded using the biotin COOH PAMAM dendrimer stain described in FIG. 7B over the conventional streptavidin phycoerythrin stain described in FIG. 7A.

FIG. 9A is a schematic representation of the steps involved in detection biotin using an anti-biotin antibody and a detectably-labeled dendrimer.

FIG. 9B is a scanned image of a spotted biotin array detected by an anti-biotin antibody conjugated to a 3DNA dendrimer.

FIG. 9C is a scanned image of a spotted biotin array detected by staining with a 3DNA dendrimer that was not conjugated to an anti-biotin antibody.

FIG. 10A is a schematic representation of the steps involved in detection biotin using streptavidin and a detectably-labeled dendrimer.

FIG. 10B is a scanned image of a spotted biotin array detected by streptavidin bound to a 3DNA dendrimer through a biotin labeled capture oligonucleotide.

FIG. 10C is a scanned image of a spotted biotin array detected by staining with streptavidin and a Cy5 labeled 3DNA dendrimer. The absence of a biotinylated bipartite capture probed fails to localize the fluorescent Cy5 3DNA dendrimer.

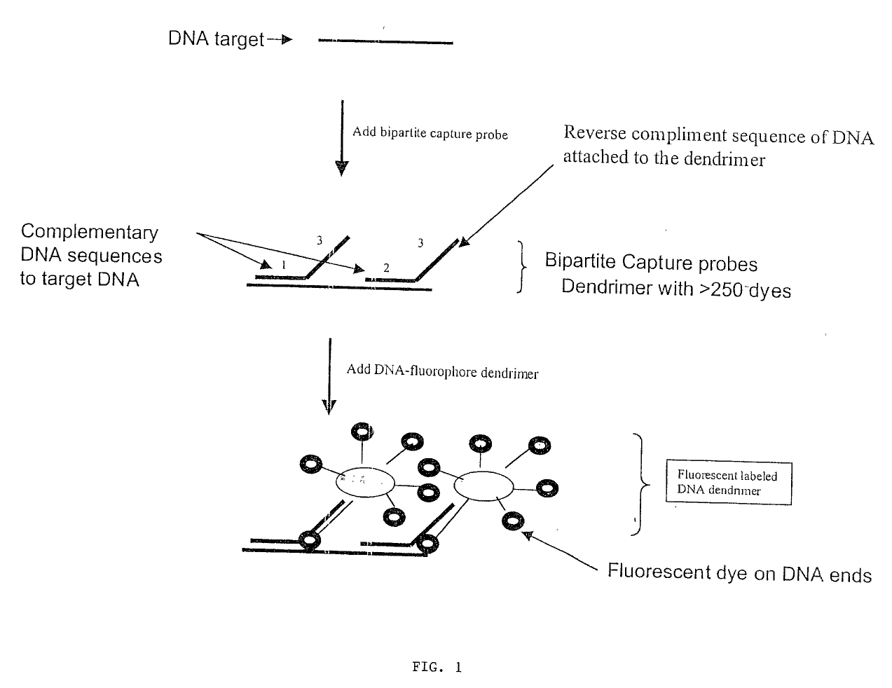

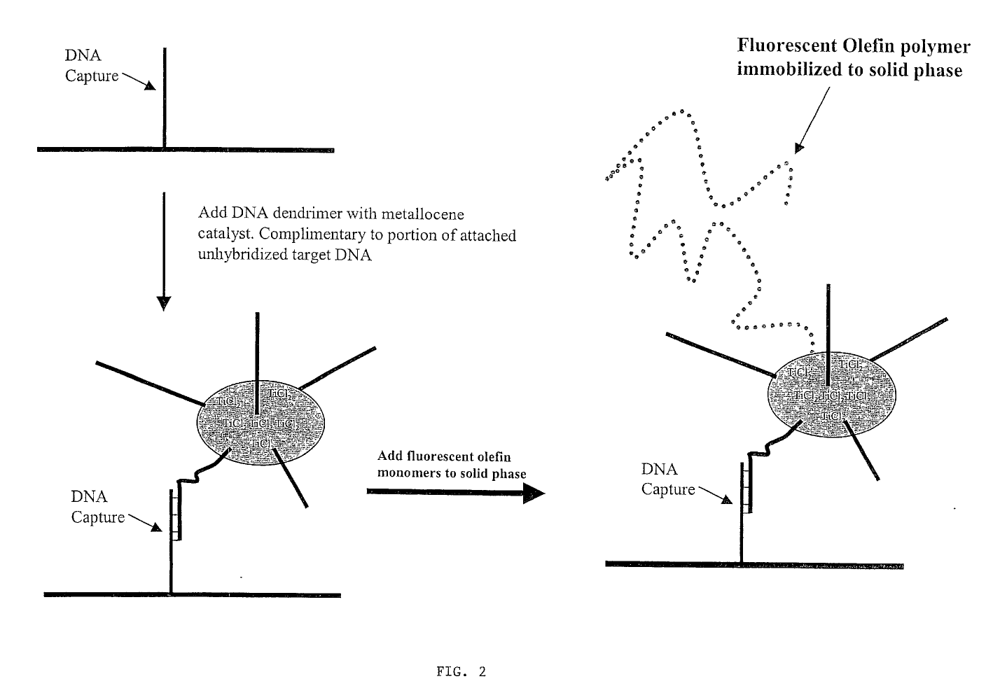

### DETAILED DESCRIPTION OF THE INVENTION

Dendrimers are a class of macromolecules or polymers that have a regular branching structure. Dendrimers are also known as cascade, starburst, cauliflower, hyperbranched, or fractal polymers. Conventional polymers, for example, polystyrene (foam packaging), polyethyleneterephthalate (PET, plastic drink bottles), and polyvinylchloride (PVC) are essentially linear polymers. When these conventional polymers are synthesized in a mixture, polymers of differing lengths having a distribution of molecular weights and sizes result. In contrast, dendrimers are generated step by step by the repetition of reactional sequences, allowing multiplication of a number of repetitive units and terminal groups. Each reactional sequence creates what is called a new generation that has a higher number of functional sites than the previous generation. When built in this stepwise manner, dendrimers are spherically shaped, porous, and have a precise weight and density. These dendrimers also have a known number of repetitive units and terminal groups. The highest generation in the polymer, (i.e., the outermost layer of the polymer) can be labeled with a detectable label, for example, a fluorescent molecule, an enzyme, or a radioactive agent if so desired. A single recognition of a target nucleic acid molecule by one detectably labeled DNA dendrimer, for example, a biotin-labeled dendrimer (which can be labeled with several hundred biotin molecules or more based on the number of generations in the dendrimer) enhances detection of the target nucleic acid molecule a corresponding several hundred times.

In addition, when dendrimers are used for nucleic acid detection according to the methods described herein, the dendrimers may also contain single stranded nucleic acid binding sequences, for example, capture probe nucleic acid binding sequences. The terminal groups of the dendrimeric polymers can be modified to add the single stranded nucleic acid binding sequence, as described herein.

Dendrimers can be formed from dendrimeric polymers, for example, poly(amido amine) (PAMAM). Example of dendrimers include, but are not limited to a cyclotriphosphazene-phenoxymethyl(methylhydrazono) dendrimer (a generation 5 dendrimer that contains 96 surface dichlorophosphinothioyls), a polypropylenimine dotriacontaamine dendrimer (which has primary amines), and a thiophosphoryl-phenoxymethyl(methylhydrazono) dendrimer (a generation 5 dendrimer that contains 48 surface dichlorophosphinothioyls). Alternatively, dendrimeric polymers can be DNA dendrimeric polymers. As used herein, a “DNA dendrimeric polymer” is a dendrimer polymer made entirely of DNA molecules. A DNA dendrimeric polymer is formed by the duplexing of DNA molecules as described, for example, by Stears et al. (Physiol. Genomics 3:93-99, 2000) and Neilson et al. (J. Theor. Biol. 187:273-284, 1997) the entire teachings of which are incorporated herein by reference, where a DNA macromolecular complex is grown out of several smaller duplex constructs. Multiple generations of DNA dendrimers can be designed based on sequence selection and hybridization of the next generation of duplex constructs to the existing DNA dendrimer. A DNA dendrimeric polymer can also contain functional groups displayed externally in the outer surface of the complex (on the highest generation). A DNA dendrimeric polymer can also be modified to contain a detectable label or nucleic acid sequences that hybridize to a target nucleic acid molecule or a capture probe.

By a “detectable label” is meant any means for marking and identifying the presence of a molecule, e.g., a dendrimer. Methods for detectably labeling a molecule are well known in the art and include, without limitation, radioactive labeling (e.g., with an isotope such as ³²P or ³⁵S) and nonradioactive labeling (e.g., chemiluminescent or enzymatic labeling or labeling with a hapten or a fluorescent molecule). Examples of fluorescent molecules that can be used as agents to detectably label a molecule include, but are not limited to CY3, CY5, ALEXA FLUOR 594, FAM, ROX, TAMRA, ALEXA 488, JOE, and fluorescein. Biotin, digoxygenin, and vitamin H are examples of haptens that can be used as detectable labels. Biotin-labeled molecules can be detected by binding to a detectably labeled streptavidin molecule or an anti-biotin antibody.

Dendrimers can be used to enhance detection of target nucleic acid molecules, as described in detail below. As used herein, by a “target nucleic acid molecule” is meant any nucleic acid molecule for which determination of presence or expression level is desired. The target nucleic acid molecule can be a DNA molecule, for example, a single stranded DNA molecule, or an RNA molecule, for example, a cRNA molecule. The target nucleic acid may be immobilized on a solid support, for example, a microarray. Examples of methods of making oligonucleotide microarrays are described, for example, in WO 95/11995, the entire teachings of which are incorporated herein by reference. Other methods are readily known to the skilled artisan.

The target nucleic acid molecule can be obtained from any sample that contains a gene expression product. Suitable sources of target nucleic acid molecules, include intact cells, lysed cells, cellular material for determining gene expression, or material containing gene expression products. Examples of such sources are tissue, cells derived from tissue, blood, plasma, lymph, urine, mucus, sputum, saliva, or other cell samples. Methods of obtaining such samples are known in the art.

In a preferred embodiment, the target nucleic acid molecule is specific for a cell phenotype. By “specific for a cell phenotype” is meant that expression of the target nucleic acid molecule correlates with a particular phenotype. Expression of target nucleic acid molecules can be used to determine, for example, the presence or absence of a phenotype in a sample, or if a candidate compound enhances or inhibits the cell phenotype in a sample. A particular cell phenotype may be determined by assessing the presence or expression level of one target nucleic acid molecule, or more than one target nucleic acid molecule, for example, two or more, five or more, ten or more, or twenty-five or more target nucleic acid molecules. Not all target nucleic acid molecules specific for a particular cell phenotype must be assessed in order to detect a cell phenotype. Similarly, the target nucleic acid molecule or set of target nucleic acid molecules specific for a cell phenotype that characterizes one phenotypic effect, for example, cell differentiation may or may not be the same as the target nucleic acid molecule or set of target nucleic acid molecules for a different cell phenotype, for example, cell death, a change from a cancerous phenotype to a non-cancerous phenotype, a change in susceptibility of a cell to a therapeutic, for example, a chemotherapeutic agent, or a change from a metastatic phenotype to a non-metastatic phenotype. Typically the accuracy of detecting a cell phenotype increases with the number of different target nucleic acid molecules that are assessed.

Currently, detection methods for use with high density microarrays often employ the use of a high affinity hapten (e.g., biotin or vitamin H) incorporated into a cRNA target by in vitro transcription (IVT) using biotin dUTP- and dCTP-labeled nucleotides. The biotin tagged IVT transcripts are then hybridized to an oligonucleotide microarray and stained with a fluorescent conjugate (e.g., streptavidin phycoerythrin (SA-PE)). The fluorescent stained targets are then quantitated using a laser scanning device and light detecting photomultiplier tube (PMT) system. This detection method is a one-color method and relies on a match to mismatch of many oligonucleotides from each target nucleic acid molecule in order to determine the presence or absence of a target nucleic acid molecule and its expression magnitude. The IVT process itself provides about a 500-fold amplification of the target nucleic acid molecule, and in general, the amount of material required for routine hybridization using microarrays is 10 μg of cRNA. In many experimental systems, however, the sample from which the target nucleic acid molecule is obtained is not large enough to yield 10 μg of hybridization material, thus obscuring the data or resulting in unreliable data. In addition, some experimental requirements may arise such that the experiment must be repeated multiple times in order to obtain proper statistical significance and noise determinations to achieve a level of confidence with the large data sets that are collected by high-density microarrays. Such experiments require many micrograms of sample. The present invention provides novel target nucleic acid detection techniques that allow enhanced detection of target nucleic acids, thus reducing the amount of sample required, and/or allowing many duplicate experiments to be obtained from a single sample.

The present invention features a number of methods that enhance detection of target nucleic acid molecules through the use of detectably labeled dendrimers. The detectably labeled dendrimers recognize and bind to target nucleic acid molecules through bridging molecules, for example, capture probes and/or streptavidin, using a number of different methods, as described in detail below.

As used herein by “capture probe” is meant a nucleic acid molecule capable of recognizing and binding, for example, through hybridization, to another nucleic acid molecule. The capture probes of the present invention may be linear or branched. When a capture probe is used to bridge a target nucleic acid molecule and a dendrimer, the probe may be a bipartite probe made up of two regions: a target nucleic acid capture region that recognizes and hybridizes to the target nucleic acid molecule, and a dendrimer capture region that recognizes and hybridizes to a single stranded nucleic acid sequence (capture probe nucleic acid binding sequence) that comprises the reverse complement of the nucleic acid sequence of the dendrimer capture region of the bipartite capture probe.

Alternatively, a capture probe of the present invention comprises a dendrimer capture region that recognizes and hybridizes to a single stranded nucleic acid sequence (capture probe nucleic acid binding sequence) that comprises the reverse complement of the nucleic acid sequence of the dendrimer capture region of the capture probe that is labeled with biotin. This type of capture probe is used to bridge a detectably labeled dendrimer to a streptavidin-bound biotin-labeled target nucleic acid molecule, as described herein.

Another capture probe of the present invention comprises a dendrimer capture region that recognizes and hybridizes to a single stranded nucleic acid sequence (capture probe nucleic acid binding sequence) that comprises the reverse complement of the nucleic acid sequence of the dendrimer capture region of the capture probe and is conjugated to an antibody that specifically binds biotin. This type of capture probe is used to bridge a detectably labeled dendrimer to biotin-labeled target nucleic acid molecule, as described herein.

### Novel Dendrimer/Nucleic Acid Conjugated Complexes

As described above, the present invention features dendrimers comprising a nucleic acid molecule, for example, a single stranded nucleic acid sequence, conjugated to a dendrimeric polymer. Polymer based dendrimers, for example, poly(amidoamine) dendrimers (PAMAM) dendrimers are not easily conjugated to DNA probes by standard chemical coupling agents because DNA complexes with the positively charged residues on the PAMAM dendrimer (Bielinska et al., Nucleic Acids Research 24:2176-2182, 1996; Bielinska et al., Bioconjugate Chem. 10:843-850, 1999; and Roessler et al., Biochemical and Biophysical Research Communications 283:124-129, 2001). Often this DNA complexation results in an insoluble complex due to maximal exposure of the hydrophobic purine and pyrimidine bases.

One novel way to solve the problem of the formation of an insoluble charged DNA conjugated PAMAM dendrimer complex is to remove the negatively charged phosphate backbone of DNA. This is accomplished by preparing peptide nucleic acid (PNA)-PAMAM dendrimers. Use of a peptide nucleic acid (PNA) construct allows one to generate nucleic acid sequences that hybridize to the dendrimer capture region of the capture probes described herein and that are chemically attached to the functional groups on the PAMAM dendrimers. The PNA can be end-labeled with a label, for example, CY3, CY5, ALEXA FLUOR 594, FAM, ROX, TAMRA, ALEXA 488, JOE, or fluorescein to provide sensitive detection of target nucleic acid molecules that bind to the labeled dendrimers using, for example, methods described herein. Such PNAs are commercially available. The PAMAM dendrimer is also commercially available, for example, from Dendritech, Inc. (Midland, Mich.) and is made from a core chemical of ethylenediamine-core poly(amidoamine).

In one embodiment, the PNA contains a sulfhydral group in the form of a cysteine as a moiety that reacts with the PAMAM dendrimer, and the PAMAM dendrimer contains several hundred primary amines (NH₂ groups) as points of attachment for the PNA. Prior to the conjugation, the PAMAM dendrimers are reacted with a hetero-bifunctional cross-linking agent capable of covalently binding primary amine groups to sulfhydral groups (e.g., N-(gamma-maleimidobutyryloxy) succinimide ester (GMBS), from Pierce Chemical Company, Rockford, Ill.) in order to activate the dendrimer. Common hetero-bifunctional crosslinking agents contain a sulfhydral reactive maleimide chemically spaced to an NH₂ reactive NHS ester. The activated dendrimer is purified from unreacted coupling agent by gel filtration (e.g., using a PD10 column) and then added to the thiol PNA containing either a dye or hapten moiety. After reaction, the complex is purified by precipitation, gel electrophoresis, or chromatography.

Alternatively, the PNA can be synthesized to contain one or more lysine groups attached to the 3′ end of the PNA and a detectable label at the 5′ end of the PNA. The NH₂ group in each lysine of the PNA is conjugated to a PAMAM dendrimer that has been modified to contain a reactive NHS ester using a homobifunctional crosslinking agent, for example, 1,4-Phenylene diisothiocyanate (Pierce Chemical Company) or other agents, such as bis(sulfosuccinimidyl) suberate (BS³), disuccinimidyl suberate (DMS), dimethyl pemelimidate•2HCl (DMP), and dimethyl adipimidate•2HCl (DMA). Prior to conjugation, the PAMAM dendrimer is activated by incubation of the PAMAM dendrimer buffer with a molar excess of the homobifunctional crosslinking agent. Next, the unreacted crosslinking agent is removed from the activated PAMAM dendrimer sphere by purification. The activated PAMAM dendrimer is then added to the lyophilized residue of the PNA containing the primary amine/lysine end group and the fluorescent moiety located on the 5′ end. The PNA-labeled sphere is then purified from free PNA and free activated PAMAM dendrimer by HPLC.

Variations in conjugation of the PNA to the PAMAM dendrimer involve converting the primary amine on either the PAMAM dendrimer or the PNA to thiol groups using 2-iminothiolane (2IT). This permits the use of heterobifunctional crosslinking agents, allowing more versatility in (1) reaction conditions; (2) extent of reaction; (3) linker arm length (when a coupling agent, for example, BS³ and other chemicals is used, the linker arm length often has to be taken into consideration; some linkers can alter solubility in a negative manner way, causing a failed conjugation while linker length also increases the distance between conjoined molecules allowing more mobility in the event that stearic factors are important); and (4) enhanced solubility of linker arms.

While removal of the negatively charged phosphate backbone of DNA, as described above, is one method of conjugating a nucleic acid molecule to a dendrimer, removal of the positive charge group from the dendrimer for nucleic acid:dendrimer conjugation is another method for conjugating a nucleic acid molecule to a dendrimeric polymer, such as a PAMAM dendrimer, while avoiding insoluble complex formation. High display dendrimers containing functional groups not positively charged are commercially available from Dendritech, Inc. (Midland, Mich.). Removal of the positive charge from the dendrimer greatly reduces the charge:charge interactions between the negatively charged phosphate backbone in nucleic acid molecules and the dendrimer. There are a number of commercially available crosslinking chemistries to enable conjugations of these dendrimers to nucleic acid molecules. Two functional groups on dendrimers that are suited for conjugation of DNA to the dendrimer are hydroxyl and carboxyl groups. Methods for conjugating dendrimers containing such functional groups to DNA are described in Examples 2 and 3.

### Enhanced Detection of Target Nucleic Acid Molecules Using Dendrimers

Detection of a target nucleic acid molecule can be enhanced through the use of detectably labeled dendrimers, which when bound to the target nucleic acid molecule provide it with numerous detectable labels, for example, haptens and fluorescent labels. A dendrimer for use in the nucleic acid detection methods of the present invention can recognize and bind to a target nucleic acid molecule in a number of different ways, as described in detail below.

1. Use of Bipartite Capture Probes for Nucleic Acid Detection

Bipartite probes present a way of bringing together and localizing two nucleic acid sequences. In the present invention, a bipartite probe is used as a means of detection whereby one portion of the probe recognizes and hybridizes to a nucleic acid sequence of interest (target nucleic acid molecule) and a second portion of the probe recognizes and hybridizes to a DNA sequence attached to a dendrimer structure. The dendrimer provides signal amplification, as it also contains detectable labels, for example, flourescent dye groups attached to the dendrimer. Thus, the bipartite probe enables recognition and detection of any desired target nucleic acid sequence.

The bipartite probe approach for target nucleic acid detection is based on recognition of the nucleic acid sequence conjugated to the dendrimer by the capture probe and is limited only by sequence selection. Several different target nucleic acid capture region sequences, each of which hybridizes to a different sequence of the same target nucleic acid molecule, may be used to generate the target nucleic acid molecule capture region of the bipartite capture probe but contain a common dendrimer capture region for enhanced signal generation. This bipartite dendrimer probe capture method is effective at detecting unpurified DNA molecules and allows versatility in the number of capture probes selected for hybridization to each target nucleic acid. In addition, since the dendrimer can be prepared with a known number of detectable labels, quantitation of target nucleic acid molecule levels is very accurate.

Detection of a target nucleic acid molecule using the bipartite probe capture method involves the following steps:

(1) A target nucleic acid molecule, for example, a cDNA molecule is immobilized on a solid phase, for example, glass. Alternatively, the target nucleic acid may not be bound to a solid support.

(2) The target nucleic acid molecule is made available for hybridization by a denaturing step, for example, by boiling in sterile water.

(3) The solid phase containing the immobilized target nucleic acid molecule is contacted with the bipartite capture probe, contained in a hybridization buffer.

(4) An incubation time is allowed and then the solid support is washed.

(5) The solid support containing the bipartite capture probe bound target nucleic acid molecule is then contacted with a detectably labeled dendrimer containing a nucleic acid sequence that recognizes and hybridizes to the dendrimer capture region of the bipartite probe, for example, a fluorescent labeled dendrimer having single stranded capture probe binding sequences conjugated to it, which is contained in a hybridization buffer.

(6) An incubation time is allowed and then the solid support is washed and scanned for fluorescent signal.

The target nucleic acid molecule that is measured or assessed using methods described herein is the numeric value obtained from an apparatus that can measure nucleic acid expression levels. The values are raw values from the apparatus, or values that are optionally re-scaled, filtered and/or normalized. Such data is obtained, for example, from a GeneChip® probe array or Microarray (Affymetrix, Inc.; U.S. Pat. Nos. 5,631,734, 5,874,219, 5,861,242, 5,858,659, 5,856,174, 5,843,655, 5,837,832, 5,834,758, 5,770,722, 5,770,456, 5,733,729, 5,556,752, the entire teachings of which are incorporated herein by reference), and the expression levels are calculated with software (e.g., Affymetrix GENECHIP software). Detection of the detectably labeled dendrimer bound target nucleic acid molecule immobilized on a microarray is done using a scanner that can detect the detectable label on the dendrimer, e.g., if the dendrimer is fluorescently labeled, the hybridization data are collected as light emitted from the labeled groups. Other arrays using fluorescent scanners and multicolor multiplex detection can be used with their appropriate fluorophores.

Two problems that the above bipartite capture probe method solves are how to screen thousands of chemical compounds for a desired ability to change a given phenotype and how to detect specific changes in cellular gene expression after exposure to a chemical compound for thousands of compounds. Currently, comparative gene expression profiles from a high density DNA microarray is used to ascertain changes in gene expression. Testing synthetic or natural chemical compound libraries as large as 100,000 or larger would require running an equivalent number (e.g., 100,000) of gene expression chips. This task is costly, time consuming, and demanding from both a data management perspective and an experimental perspective, and therefore it is impractical to run a new gene chip expression profile for each chemical compound being tested. A detailed description of the use of this method to detect cell phenotype changes is provided in Example 4.

2. Biotin DNA Oligonucleotide with a Streptavidin Bridge for Dendrimer Display

A target nucleic acid molecule can be detected by a three step process involving providing a biotin containing target nucleic acid molecule, for example, a cRNA target molecule that is immobilized on a support (as described, for example, in WO 95/11995, the entire teachings of which are incorporated herein by reference). The target nucleic acid molecule is exposed to streptavidin as a bridge for capture of a biotin containing DNA oligonucleotide whose sequence is the reverse compliment of a biotin-labeled dendrimer, for example, a DNA dendrimer or a dendrimer comprising nucleic acid molecule conjugated to a non-DNA dendrimeric polymer, and is therefore capable of hybridizing to the dendrimer. The biotin-labeled dendrimer also displays a large number of additional biotin molecules for staining with phycoerythrin as described by Coller et al. (Proc. Natl. Acad. Sci. USA 97:3260-3265, 2000, the entire teachings of which are incorporated herein by reference). Streptavidin contains four high affinity binding sites. Therefore, upon recognition and binding of biotin (in the target nucleic acid molecule) to streptavidin, three additional available unbound biotin binding sites exists on streptavidin. This target nucleic acid detection method utilizes these unbound binding sites as a bridge to capture a biotin-labeled nucleic acid capture probe. The capture probe comprises a sequence specific to the DNA sequence in a biotin-labeled dendrimer described above. The biotin-labeled dendrimer displays a large number of biotin molecules for enhanced detection (up to several hundred biotins). The dendrimer, and therefore the target nucleic acid molecule is then detected using standard methods, for example, those described above.

3. Anti-biotin Antibody-DNA Conjugate for Detection of Target Nucleic Acid Molecules (Immunodendrimer Capture)

Another method for detection of target nucleic acid molecules using dendrimers employs the use of an anti-biotin antibody conjugated to a nucleic acid capture probe. The nucleic acid capture probe comprises a nucleic acid sequence that hybridizes with its complimentary DNA sequence in a dendrimer, for example, a biotin-labeled DNA dendrimer or a dendrimer comprising a nucleic acid molecule conjugated to a non-DNA dendrimeric polymer, as described herein. The anti-biotin antibody provides the basis for attraction of the biotin-labeled dendrimer. This method is unlike the streptavidin bridge method described above in that it uses a direct covalent coupling of the capture probe to the antibody. This method has been put into practice, as described in Example 5. This method can also be used for amplified detection of single nucleotide polymorphisms on a solid phase microarray. Using single base extension (SBE) one can extend a single base tagged with a hapten into any oligonucleotide sequence and then detect this single base extension with the DNA capture dendrimer.

4. Detection of Target Nucleic Acid Molecules Using a Biotin-Labeled Dendrimer

In another target nucleic acid molecule detection method, streptavidin is used as a bridge to co-localize a dendrimer containing many biotins to a single biotin in the target nucleic acid molecule, for example, a target cRNA molecule. The dendrimer is derivatized at its functional groups with a biotin molecule spaced by a hydrocarbon linker. This biotin-labeled dendrimer can be synthesized, for example, from an NH₂ reactive NHS ester biotinlyation kit (commercially available from Amersham Pharmacia Biotech (APB)) and then purified from unreacted biotin using a PD10 gel filtration column. The biotin dendrimer complex is then stained with SA-PE as described by Coller et al. (supra), with the following two steps added prior to the procedure of Coller: (1) adding streptavidin first for 30 minute incubation in 1× MES buffer; then (2) adding biotin PAMAM dendrimer in 1× MES binding buffer. This method has been put into practice on microarrays and has shown to provide improved signal amplification. The construct used was a biotin PEO₄ coupling agent (Pierce Chemical Company) and a generation 7 PAMAM dendrimer (Sigma Aldrich).

5. Detection of Target Nucleic Acid Molecules Using Nested Dendrimers

An additional benefit of DNA dendrimers is their use in multiple complex formation (nested dendrimers), which consequently provides enhanced detection. This enhanced detection arises from both the specificity of the DNA sequence and the high display of the DNA sequence attached to any dendrimer. A nested detectably labeled dendrimer display method can be used to detect a target nucleic acid molecule. In this method, the sequence of a first DNA dendrimer or dendrimer comprising nucleic acid molecule conjugated to a non-DNA dendrimeric polymer is hybridized to a second dendrimer containing biotins for display and subsequent reaction to SA-PE. The first dendrimer comprises a mixture of two capture sequences such that one sequence hybridizes to the nucleic acid capture probe in a bridge complex as described above, and a second sequence that hybridizes to a second detectably labeled dendrimer. The first dendrimer provides an added level of amplification by providing many potential hybridization sites for localization of the secondary biotin display dendrimer.

6. Use of Light Harvesting Cationic Conjugated Polymers to Enhance Emission Properties of DNA or PNA Dendrimer Complexes

Cationic conjugated polymers can be used to enhance emission properties of fluorescent moieties (fluors) attached to peptide nucleic acid-DNA duplexes (Gaylord et. al., PNAS 99(17):10954-10957, 2002). Cationic polymers are attracted toward the negative charges of the phosphate backbone of DNA. Using a neutrally charged fluorescently labeled peptide nucleic acid (PNA) sequence (PNA-fluor) hybridized to a DNA target sequence one skilled in the art can co-localize a positively charged “light harvesting” conjugated polymer. The polymer, once bound to the PNA-DNA duplex, results in an enhanced fluorescence emission by a process commonly known a Förster Resonance Energy Transfer (FRET). The FRET mechanism is a coulombic inductive process wherein the excitation dipole of one light absorbing species emits an electronic wave capable of inducing a dipole separation (“excitation”) in neighboring molecules. The optical “absorbance” properties of the cationic polymer are chosen to favor the excitation spectra of the fluor attached to the PNA molecule.

In the present invention, the above-described process may be employed wherein a fluorescently labeled DNA or PNA dendrimer is used to contact a nucleic acid “target” sequence localized onto a surface such as a microarray. The nucleic acid dendrimer complex is used as a high display molecule capable of localizing several hundred detectable probes such as fluorescent molecules, for example, the dendrimers described herein. The amplified detection of the nucleic acid probe may be even further enhanced by subsequent staining/application of a conjugated cationic polymer. The specifically hybridized DNA dendrimer or PNA dendrimer complex is then exposed to a cationic conjugated polymer which has an absorbance spectra matching the excitation spectra of the fluor in the nucleic acid conjugated dendrimer. The extent of fluorescence enhancement is largely dependent on the spectral integral of overlap between the fluor and the conjugated cationic polymer. One example of such a cationic conjugated polymer is poly (9,9-bis(6′-N,N,N-trimethylammonium)hexyl)fluorene phenylene with iodide counter ions.

In the present invention, a PNA-fluor dendrimer construct is employed to detect a spotted gene on a microarray. Subsequent hybridizations of unused PNAs on the dendrimer with “decorator” DNA-fluor oligos, as described herein, can be made for additional amplifications. These fluor:PNA-fluor:DNA-dendrimers can then be further amplified by contacting the final complex with a conjugated cationic polymer which has optical properties favorable for FRET based sensitization of those fluors contained in the nucleic acid dendrimer complex.

7. Use of Nucleic Acid Dendrimer Conjugates for Enhanced Multiplex Detection on Surface Plasmon Resonance Systems

Surface plasmon resonance (SPR) is a technique that is widely used to detect binding events on a solid phase surface (Sigmundsson et al, Biochemistry 41(26):8263-8276, 2002 and Myszka et al., Biophy. J. 75(2):583-594, 1998). These binding/recognition events are based on changes in refractive index on a metal-glass surface. The metal provides a medium favorable for generation of a plasmon resonant evanescent wave field, which provides an electronic field capable of detecting subtle changes in refractive index due to mass changes near the field. Since dendrimers are spherical polymers of well-defined size and molecular mass, the attachment of sequence recognizing nucleic acids or hapten binding elements to the synthetic dendrimer polymer can provide a mechanism for recognition that provides an additional mass change in an SPR detecting device. Hence, the attachment of DNA, PNA, or any nucleic acid analogue or hapten (e.g., biotin, protein, antibodies, peptides, polysaccharides) to a dendrimer as described herein can be further extended beyond fluorescent and chemiluminescence detection to also include those detection systems that rely on mass/refractive index changes.

Metals/ions, haptens, antibodies, peptides, small molecules, nucleic acids and any other molecule capable of molecular recognition can be attached to a polymer and utilized as a “signal enhancer” by virtue of its relatively large molecular weight. Dendrimers, for example those described herein, provide a means of introducing a uniform elevation in molecular weight. The introduction of DNA sequence recognition, for example, as described herein, also enables a high degree of multiplex detection and by various size distributions. For example, one could label a generation 3 dendrimer with one sequence and a generation 6 dendrimer with another nucleic acid sequence and yield a two mass detection enhancement range.

8. Signal Amplification by Use of Photopolymerizable Crosslinkers Attached to Detectable Dendrimer Constructs

As described herein, dendrimers provide a means of localizing many fluorophores for each specific recognition. Another application of dendrimers, which may also enhance the localization of high display fluorophores, is the use of a duel display dendrimer having both fluorophores and photocrosslinkable chemicals. These photocrosslinkable chemicals are useful in that they enable one the ability to amplify signal by polymerization of additional dendrimers. One such chemical capable of performing this task is a member of the cinnamate family. Members of this family of compounds undergo a 2+2 photo-cylcoaddition reaction when the molecule is irradiated with the appropriate wavelength of UV light.

The present invention features a signal amplification reagent wherein a PAMAM dendrimer structure polymer is chemically conjugated to a photocrosslinkable chemical and a detecting moiety. For example, the dendrimer can contain a DNA sequence capable of recognizing another DNA target immobilized on a DNA microarray. The DNA sequence can contain a detectable label, such as biotin or a fluorophore. In addition to the fluorescent or hapten labeled DNA the dendrimer can also contain a covalently bound photocrosslinkable chemical such as cinnamylidene acetyl. Cinnamylidene acetic acid is conjugated to the functional NH₂ and COOH groups on the dendrimer by use of a chemical coupling agent such as 1-Ethyl-3-(3-Dimethylaminopropyl)carbodiimide Hydrochloride (Pierce Chemical Company).

Alternatively, a first recognition dendrimer can be used to localize the cinnamylidene group and a secondary stain can be applied that is a combination of detectable fluorophore or hapten and photocoupling group. Upon excitation with a standard 450 W medium pressure UV lamp (a filter can be used to cutoff wavelengths below 300 nm) polymerization occurs. Alternatively, excitation/photopolymerization can be achieved with either a nitrogen laser or a HeCd laser. The kinetics of the photopolymerization can be controlled by concentration of the stain, temperature and irradiation time. The use of photopolymerization of polymers is known to one of skill in the art (Andreopoulos, et. al., J. Am. Chem. Soc. 118:6235-6240, 1996).

### Secondary Emission Enhancement “Decoration” of Dendrimer Capture

The bipartite probe described above enables retention of a dendrimer containing multiple DNA oligonucleotides (capture regions) with fluorescent (or detectable labels i.e., radioactive, chemiluminescent, quantum dots, or resonance light scattering particles) end labels on essentially any solid phase. Each dendrimer can contain up to several hundred and even thousands of detectable labels. Nonetheless, upon completion of the initial binding reaction the unused oligos are still available for subsequent binding reactions. These unused oligos can be used to retain reverse complimentary DNA that with further introduction of additional detectable labels can enhance the already amplified DNA end labels which are localized to the target on solid phase. If all oligos were fully available then decoration of unused DNAs on the dendrimer would add an almost doubling of detection. For example, if a dendrimer had 500 fluorescent DNA end-labeled attachments and only required one probe sequence to retain the complex, then a subsequent total decoration would enable an additional 499 reverse complimentary with single end-labeled DNAs to be added to the recognition event. Also the end labels may require specific separation distances to avoid the possibility of any inner filter effects. Inner filter effects often occur when one adds a sufficiently high concentration of absorbing species to a solution (solid phase) that an attenuation of possible excitation of fluorophores occurs.

### Single Oxygen Released Light Emitting Decorator Coupled with Fluorescent Molecule Decorators

DNA dendrimer conjugates can contain multiple DNA sequences. This provides the added versatility of using relay emission systems. One such commercial system is that of Packard Biosciences. Beaudet et al. (Genome Research 11:600-8, 2001) have demonstrated the use of this powerful light emitting system to detect single base polynucleotide changes in DNA sequences. In this method, phthalocyanine (donor #1) upon absorption of light at 680 nm will release a singlet oxygen. If an acceptor molecule is used such as thioxene on another bead particle located nearby (less than 200 nm) one should expect to see a bright emission at 370 nm. This emission can be coupled with other fluorophores located near by to raise the emission to the 520-620 range. The other fluorophores coupled to this system are anthracene and rubrene. In addition, Beaudet et al. have developed a secondary donor called napthalocyanine, which absorbs at around 780 nm has been developed. The extent of light emission coupled with low background noise makes this detection system many times more sensitive to most fluorescent labels. A complex system of dendrimers can be constructed in which the first dendrimer recognition localizes many singlet oxygen donor group molecules, while the second nested dendrimer contains the appropriate ratio of DNA end labels that include the same emission relay as used by the above described system.

Dendrimers as Nanoreactors: DNA Dendrimers as a New Composition for Encapsulation of Polymer Catalysts for Enhanced Detection on Solid Phase

Dendrimer structures can be used as a tool for amplifying signal on a solid phase, by using the porous nature of the dendrimer to encapsulate a polymer catalyst. The dendrimer may be derivatized or coupled to a detectable label and hence provide a localization for the initiation of a polymer reaction. The polymer reaction yields a polymer capable of being detected by means such as spectroscopy or electrochemical or fluidic control. Metal particles can be encapsulated into a dendrimer structure and used as a catalyst for chemical reactions (Crooks et al., Acc. Chem. Res. 34:3, 2001, the entire teachings of which are incorporated herein by reference). For example, dendrimer structures can be coupled to DNA and employed for detection of nucleic acid sequences on a solid support. A polymer stimulating catalyst (metal or organic) is embedded into a dendrimer structure that contains a detecting component such as DNA, RNA, PNA or a peptide/antibody structure. The detecting dendrimer-embedded catalyst recognizes the target of interest. After recognition, the complex is exposed to a co-monomer solution, and a polymer reaction proceeds from the dendrimer structure. The catalyst can be of an organo-metallic nature or for a photochemical reaction. The dendrimer acts as a nanoextruder or nanoreactor. The polymer structure is detectable by using monomers that contain fluorescent or chemiluminescent side groups or functional groups that are attachable by reactive coupling agents such as primary amines, aldehydes, sulfhydryl groups, or COOH. Radioactive monomers can also be used for detection to provide an amplification enhancement.

### Assembly of Metal Encapsulated Dendrimers as an Ultrasensitive Nanostructure for Analyte Detection

Crooks et al. (supra) have demonstrated the ability to encapsulate metal ions into the internal pores/cavities of starburst dendrimers. One intriguing possibility is the use of these metal encapsulated dendrimer particles in super macromolecular structures of defined geometry and radius for use as a light scattering reagent. U.S. Pat. No. 6,214,560 describes colloidal particulate synthesis methods to achieve unique size particles capable of scattering light at a defined wavelength, and demonstrates that the emitted light of a single metal nanoparticle can be greater than one million fluorescein fluorophores. This degree of light intensity is of great utility in microarray experiments and should by far exceed the detection performance of other methods such as rolling circle DNA amplification, tyramide amplification, DNA dendrimer amplification and quantum dot emission. This amplifying tool can be coupled to other amplifying methods for increased performance and enable single molecule counting. For example, detection of SNPs in spotted genomic DNA might well be in reach with such a system. However, one current difficulty in the field has been the ability to synthesize a particle of well-defined radius. The radius of the particle defines the intensity of the scattered light from the particle. Therefore, methods that enable synthesis of light scattering particles of well-defined radius, should be of great importance to the field. An especially important application is that of gene expression arrays wherein one attempts to use multicolor detection to compare to states of gene expression. Thus, having a two color detection system with relatively comparable intensity will advance the field.

In the present invention, several methods are proposed by which one can assemble discrete unique size particles either in solution or in situ. The light scattering particles described in this invention mainly comprise the use of nanoporous dendrimer polymers for the incorporation of metal compounds. There is a wide selection of dendrimer polymer sizes available whose radius and pore size are defined according to their generation of growth.

### Protein-Hapten Assisted Macromolecular Assembly of Metal Containing Dendrimers

In this method, a dendrimer polymer of desired diameter is derivatized externally with functional groups to contain a high affinity hapten, such as biotin. Provided that the particle maintains its solubility, the polymer is soaked with the desired metal, such as gold. The complex is purified away from unincorporated metals by gel filtration or any desired chromatography such as HPLC. The metal encapsulated dendrimer may be used to grow a light scattering particle directly on a solid surface using a streptavidin bridge between the analyte on the solid surface and the detecting moiety. Once bound to the solid surface one can then begin a layering process whereby biotin metal dendrimers, then streptavidin, then biotin metal dendrimers are added until the appropriate degree of particle size is achieved.

### DNA Assisted Macromolecular Assembly of Metal Containing Dendrimers

Another approach for assembling metal containing dendrimers is to employ the specific sequence hybridization potential of DNA to direct the size of the metal encapsulated dendrimer. In this experiment, a metal encapsulated dendrimer is externally derivatized with either a PNA or DNA to form a DNA sequence affinity reagent. This reagent can be used to either detect a specific DNA sequence spotted on an array or to directly control particle growth size in solution. The DNA displayed externally is used to attract a second metal encapsulated dendrimer for controlled particle size growth either in situ or in solution. Multiple DNA sequences attached to a single metal encapsulated dendrimer can be used to achieve complex layering of multiple dendrimers of various sizes and layers.

One special variation of this is the use of thiol DNA to act as a covalent attachment site to gold metals in particular. For example, Mirkin et al. (Nature 382:607-609, 1996) demonstrated the use of thiol DNA to assemble gold particles for controlled aggregate formation. Similarly, dendrimers derivatized with thiols externally or via DNA can also be used to attract gold particles into a dendrimer structure.

### Enzymatic Amplification of Nucleic Acids on a DNA-dendrimer for Improved Detection of Analytes

As described herein, nucleic acid conjugated dendrimers enable the localization of many common or mixed nucleic acids. The nucleic acids can be normal or modified DNAs, RNAs, PNAs or locked nucleic acids (LNAs). Localization of the DNA dendrimer to a solid support can occur by a number of means, such as nucleic acid hybridization, hapten recognition (e.g., biotin, digoxygenin) or immunoreactive recognition (for example, a DNA capture sequence for the DNA dendrimer that is conjugated to an antibody). As described herein, DNA detection can occur by the introduction of detectable moieties such as fluorescent or chemiluminescent molecules to the DNA that is attached to the nucleic acid-dendrimer. Enzyme linked immunosorbent assays (ELISA) assays are often based on fluorogenic or colorometric product generation by enzymes conjugated to an antibody. Another nucleic acid detection application for nucleic acid dendrimers is the use of enzymatic amplification reactions to further enhance detection.

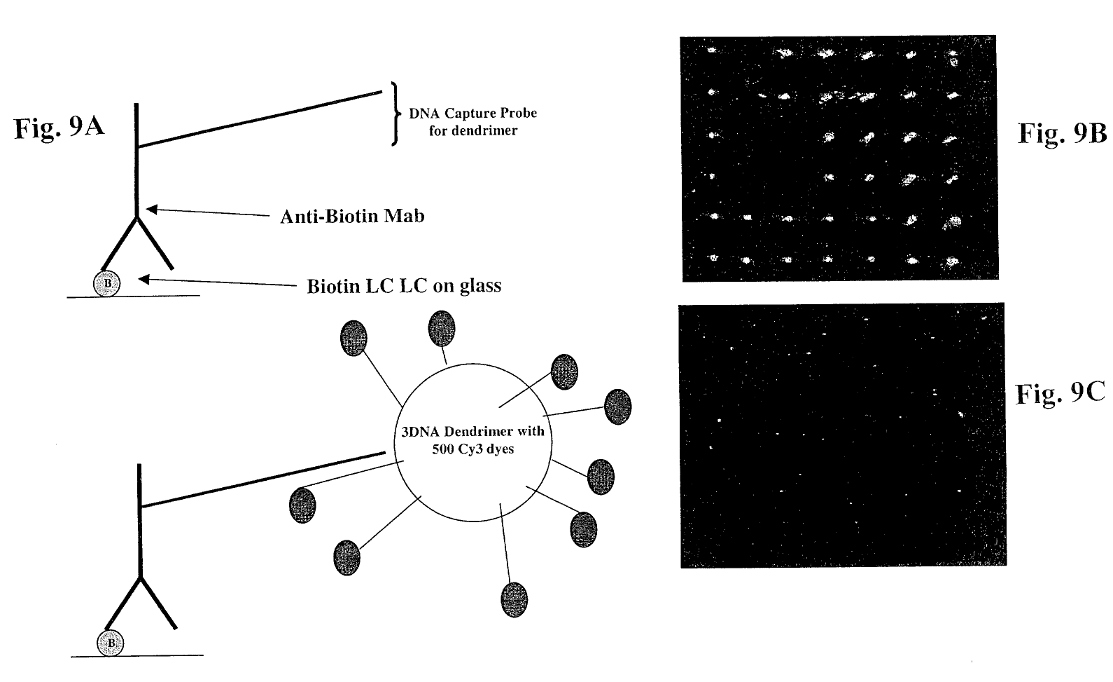

The present invention also features a method of co-localizing a nucleic acid conjugated dendrimer using an antibody (as shown, for example, in FIG. 9). The co-localized nucleic acid can then be used in a subsequent PCR reaction or in an in vitro transcription reaction. Zhang et al. (PNAS 98:5497-5502, 2001) has shown the ability to attach a T7 RNA promoter directly to a monoclonal antibody and then use T7 RNA polymerase to generate large amounts of cRNA off the analyte bound antibody. Immuno-PCR (Sano et al., Science 258:120-122, 1992) utilized a DNA attached to an antibody. The DNA-antibody conjugate was then used to detect an analyte either in solution or on a solid phase. The DNA on the antibody was then used as a substrate for a DNA polymerase chain reaction (PCR), and indicated binding of a substrate by the DNA antibody conjugate. The immuno-PCR reaction gave a 10⁵ fold increase in sensitivity over conventional alkaline phosphatase ELISAs. This process is exponential and requires thermal cycling. The work of Zhang et al. (supra) has shown that employing T7 RNA polymerase can use a linear isothermal process. Other known nucleic acid amplifying reaction methods can be used to amplify DNA or RNA attached to an antibody. In addition, other RNA polymerases such as Qbeta replicase, SP6, and T3 can also be used to amplify DNA or RNA substrates attached to an antibody.

Nucleic acid dendrimers can be used to amplify DNA or RNA, using methods similar to those of Zhang et al. and/or Sano et al., however, the nucleic acid dendrimer provides a unique opportunity to provide even more amplification potential to an enzymatic nucleic acid amplification reaction by virtue of its ability to localize large numbers of putative substrates. The amplicon products of both immuno-PCR and the T7 RNA polymerase antibody reaction (IDAT) are released into solution and visualized on a gel. In the case of T7 RNA polymerase, the single stranded RNA products can be retained on a solid phase in which the reverse compliment of the sequence is attached to it.

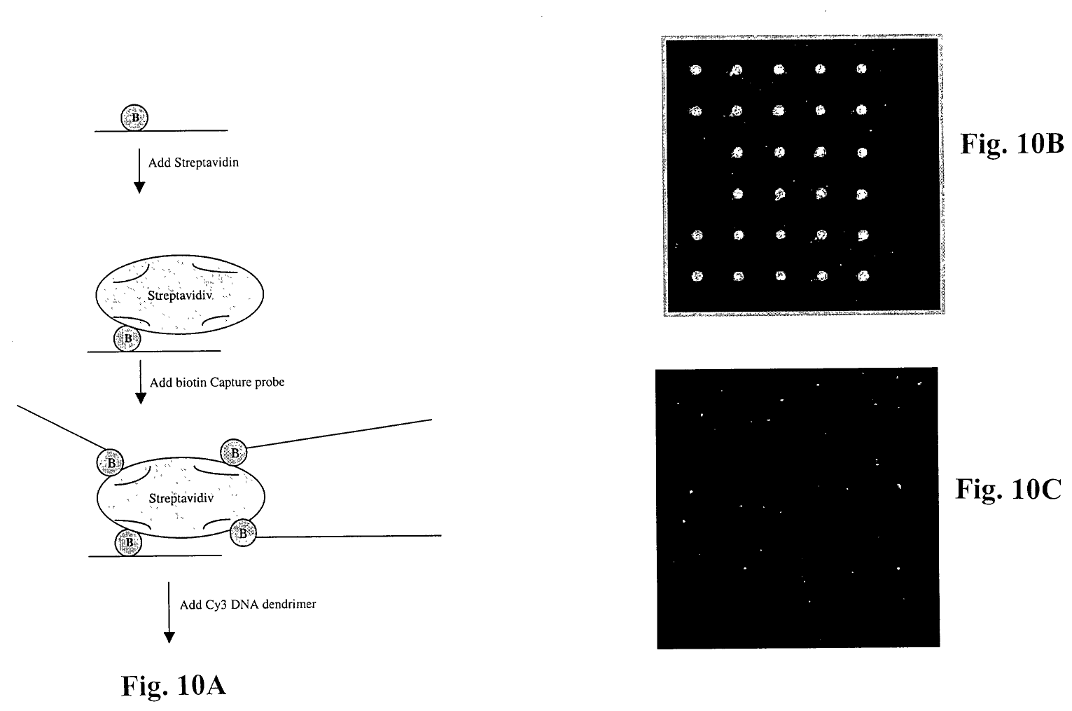

The nucleic acid dendrimer can be localized to an antibody reaction or hapten binding reaction. The dendrimer can be immobilized on a solid phase as described herein and as shown in FIGS. 9 and 10. For example, the a nucleic acid capture probe can be chemically conjugated to an antibody or to an aptamer. Alternatively, a biotin/streptavidin techniques (as described herein) can be used to attach the antibody to the dendrimer. When the antibody-DNA conjugate binds to its target, it displays a localized DNA or PNA or LNA capture probe. The DNAs in the dendrimer are complimentary to the sequence on the antibody-nucleic acid conjugate and thus allows one to capture the nucleic acid dendrimer. The DNA or RNA sequences on the dendrimer are then primed with PCR primers or are duplexed and allow for T7 RNA polymerase generation of cRNA. The amplicons of the enzymatic reaction can then be run on a gel or quantitated by real-time fluorescent nucleic acid stain and be quantitated to demonstrate antibody specific detection. The additional nucleic acid substrates on the nucleic acid dendrimer will provide significant enhancement in detection over the standard DNA-antibody reactions.

The invention will be further described with reference to the following non-limiting examples. The teachings of all the patents, patent applications and all other publications and cited herein are incorporated by reference in their entirety.

### EXAMPLES

### Example 1

### Preparation of PNA-PAMAM Dendrimers

An 18 mer PNA with one or more lysine groups attached on the 3′ end of the PNA and a Cy5 dye at the 5′ end is conjugated to a PAMAM dendrimer. The lysine group contains a positive charged NH₂ group for conjugation with an NHS ester on the PAMAM dendrimer. The PAMAM dendrimer is conjugated to the 3′ end-labeled lysine PNA-Cy5 using a homobifunctional crosslinking agent, for example, 1,4-Phenylene diisothiocyanate (Pierce Chemical Company) or other agents, such as Bis(sulfosuccinimidyl) suberate (BS³), Disuccinimidyl suberate (DMS), Dimethyl pemelimidate•2HCl (DMP), or Dimethyl adipimidate•2HCl (DMA). Prior to conjugation, the PAMAM dendrimer is activated (derivatized) by incubation of the PAMAM dendrimer in 0.1 M KPO4 (pH 7) buffer with a 50 molar excess of any one of the homobifunctional crosslinking agents for one to two hours at room temperature. Next, the unreacted crosslinking agent is removed from the activated PAMAM dendrimer sphere by passage over a G25 column (PD-10) followed by Microcon YM10 filtration. The activated PAMAM dendrimer is then added to a microcentrifuge tube containing the lyophilized residue of the PNA containing the lysine/primary amine end group and the fluorescent moiety located on the 5′ end. The reaction can be carried out, for example, for 2 hours at room temperature, to overnight at 4° C. The PNA-labeled sphere is then purified from free PNA and free activated PAMAM dendrimer by HPLC using, for example, DEAE with a reverse phase column (C4).

### Example 2

### Conjugation Between DNA Containing an End-Labeled Primary Amine and a COOH Containing Dendrimer

1-Ethyl-3-(3-Dimethylaminopropyl) carboiimide hydrochloride (EDC), commercially available from Pierce Chemical Company (Product #22980) is used to couple primary amines to carboxyl groups. This coupling reagent is combined with the COOH display dendrimer to form an activated dendrimer. The COOH dendrimer is reacted with EDC for one to two hours and then passed through a PD10 gel filtration column. The void column eluant is activated COOH dendrimer. This activated dendrimer is then added directly to a solution DNA or a pellet containing primary amine end-labeled DNA. The primary amine can be internally or end-labeled with a detectable label. The DNA can also contain a fluorescent or hapten moiety on the end opposite the conjugating primary amine group. The conjugation takes place over two to several hours at room temperature and then the mixture is separated by HPLC.

### Example 3

### Conjugation Between DNA Containing an End-Labeled Thiol (SH) Group and an —OH Containing Dendrimer

N-[p-Maleiomidophenyl]isocyanate (PMPI), commercially available from Pierce Chemical Company (Product #28100) is used to couple DNA containing an end-labeled thiol group (SH) to a hydroxyl group contained in a dendrimer (an —OH dendrimer). This coupling reagent is first combined with the —OH dendrimer, to form an activated dendrimer. The —OH dendrimer is then reacted with PMPI for one to two hours at room temperature and then passed through a PD10 gel filtration column. The void column eluant is activated —OH dendrimer. This activated dendrimer is then added directly to a solution of thiol end-labeled DNA or a lyophilized pellet containing thiol end-labeled DNA. The DNA can also contain a fluorescent or hapten moiety on the end opposite the conjugating thiol group. The conjugation can take place over several hours at room temperature and then the mixture is separated by HPLC.

### Example 4

### Use of Bipartite Capture Probes For Detection of Genes Unique to a Particular Cellular Phenotype

A set or sets of genes were determined as specific for a given cell phenotype for a given cell line by first using a gene expression microarray. Targets were selected by selecting genes that were activated upon differentiating the acute myeloid leukemia (AML) cell model HL60 into non-cancerous monocytes or neutrophils upon treatment with phorbol 12-myristate 13-acetate (PMA) for differentiation into monocytes or all-trans retinoic acid (ATRA) for differentiation into neutrophils. The entire gene profile on a high density array was used to depict the upregulated genes for the non-cancerous phenotype. For monocyte differentiation, Interleukin 1 receptor antagonist (IL1RN; GenBank Accession No. X53296) and Secreted Phosphoprotein 1 (SPP1; GenBank Accession No. U20758) were selected as target genes to be evaluated for expression using the methods described below, as these genes were up-regulated in HL60 cells in response to PMA. For neutrophil differentiation, Orosomuciod (ORM1; GenBank Accession No. X02544) and 47 kDa autosomal chronic granulomatous disease protein gene (NCF1; GenBank Accession No. M55067) were selected as target genes to be evaluated for expression using the methods described below, as these genes were upregulated in HL60 cells in response to ATRA.

The determination of whether a gene is specific for a given cell phenotype is essentially binary, such that the gene is either expressed or not expressed in a given phenotype. Whether a gene is “expressed” is determined based on whether the level of expression meets or exceeds a preset expression level. The expression level of a gene that is “not expressed” is not necessarily zero, but is less than the minimal expression level required to be “expressed.” This discrete partitioning of gene levels greatly improves the ability to determine if a gene is expressed or not expressed but is not restrictive.

HL60 cells (from a model cell line for acute myeloid leukemia) were grown in a high throughput method (384 well format) and challenged by introduction of PMA (at nanomolar concentrations for 24 hours, for differentiation into monocytes), or ATRA (at nanomolar concentrations for 48-72 hours, for differentiation into neutrophils) that would induce cell differentiation, as confirmed by phenotypic evaluation. The mRNAs of challenged cells in each well were extracted by a commercially available oligo dT coupled 384 well plate (Pierce Chemical Company) and cDNA was made from the mRNA template using methods standard in the art. The newly synthesized cDNAs were covalently attached to the well via the oligo dT primer attached to the well plate and served as an amplifying template for production of PCR products. Primers specific to the target genes for each phenotypic state (ILRN1, SPP1, ORM1, and NCF1) as well as a control gene, glyceraldehyde-3-phosphate dehydrogenase (GAPDH), whose expression is not altered by PMA or ATRA were added to each well and amplicons for these genes were obtained by PCR reaction using standard conditions as suggested by the manufacturer (Sequenom, San Diego, Calif.).

The presence or absence of the upregulated target genes (ILRN1, SPP1, ORM1, and NCF1) after exposure of the cells of a given well to a chemical compound was determined by spotting unpurified PCR amplicons from the cDNA preps from each chemical query onto a microarray. Briefly, the PCR amplicons were mixed with a spotting buffer (e.g., 5.5 M NaSCN) and then spotted via microarray technology quill pin onto a glass surface (other spotting means may be used, for example, ring/pin tool, solid pins or piezo electric deposition, described, for example, by Schena (Microarray Biochip Technology. Biotechniques Books Division. 2000. Eaton Publishing, Natick, Mass., the entire teachings of which are incorporated herein by reference). The glass surface was derivatized with aminosilane (although other slide coatings are possible substitutes such as polylysine or aldehyde silane). The spotted unpurified PCR amplicons were then immobilized onto the glass either by baking or UV crosslinking. The spotted immobilized PCR amplicons were then boiled in sterile water for two minutes to denature the PCR duplex into hybridizable single stranded DNA (ssDNA).

The genes specific for each phenotype were detected by a two step staining (hybridization) procedure as follows. The slides were pre-blocked slides 2× SSC/0.02%SDS/BSA for 1 hour at 45° C. Next, a 2× SSC/0.02%SDS/BSA solution containing at least a 1 micromolar amount of each bipartite probe for each gene was applied to the slide. Typically 4 probes were used per gene. The final volume for a 24 pin array (half of a microscope slide 1 inch by 3 inches) requires 30 μL. The solution was incubated for 40 minutes at 45° C. Next, a cocktail mix of each color of fluorescent labeled 3DNA dendrimer was added in the appropriate concentration provided by the vendor (2.5 μL per slide). The fluorescent 3DNA dendrimers were brought up to 30 μL in 2× SSC/0.02%SDS/BSA and incubated for 0.4 hours at 45° C. The slides were then washed and spun dry in a centrifuge and scanned immediately. Between each step, the slides were washed according to the following procedure. The slides were vigorous plunged in a 2× SSC solution (sodium citrate sodium chloride, pH 7.2) with 0.2% SDS for 6 minutes, followed by washing for 3 minutes in 2× SSC, followed by an additional 3 minutes of washing in 2× SSC, followed by washing for 1 minute in 0.2× SSC. After the final wash step, the slides were dried dry by nitrogen air gun or centrifugation.

The IL1RN, SPP1, ORM1, NCF1 and GAPDH genes were detected by fluorescence dendrimer stain. GAPDH was used as a positive control for the process and was common to both phenotypes in roughly equal expression levels. Each microarray also contained a control plate having the unpurified multiplexed PCR amplicons for the genes of interest from cells that (1) were not chemically challenged and represent the untreated and undifferentiated phenotype; or (2) were chemically challenged (with PMA or ATRA) and represent the differentiated phenotype. More than one differentiated phenotype can be assessed on a single microarray element. The undifferentiated controls provided a fluorescence threshold level from which expression levels could be determined. A control gene common to both phenotypes (GAPDH) and at comparable levels was used as a positive control to assess that the individual query was made. This measure aided in the avoidance of false negatives that may arise by either failed spotting or PCR. Ultimately, a determination of whether the target genes were expressed or not expressed was made. Other useful controls were (1) spotted capture probes, to assure that staining and dendrimer signals are normal; (2) the purified gene of interest; (3) grid alignment controls; and (4) blank wells to avoid carry over from certain quill pen spotting technology.

The assay without significance optimization gave a specificity of 99.5% and sensitivity of 87%. Specificity and sensitivity were calculated using the HL60 cells left undifferentiated as negative controls and PMA differentiated samples as positive controls. The ratio percent of the negatives found (AML spotted on array and stained with bipartite method) over total true negatives was 99.5%. The sensitivity was calculated as the number of positives found per total true positives, as assayed by phenotypes inspection of the cells, PCR amplification of the upregulated genes, and SBE analysis and/or measurement of the readout. Sequenom PCR reactions and gel assays were used to confirm these numbers. The specificity and sensitivity can be increased by using multiple bipartite probes for each gene of interest and multiple color dendrimers.

### Example 5

### Anti-biotin Antibody-DNA Conjugate for Detection of Target Nucleic Acid Molecules (Immunodendrimer Capture)

To demonstrate immunodendrimer capture, the following methods were carried out. One mg of Anti-Biotin IgG mouse monoclonal Antibody (mAb) was obtained from Jackson Immunoresearch Labs (West Grove, Pa.; product #200-002-096, Lot 50190). The DNA sequence for capturing a CY3-labeled 3DNA dendrimer stain molecule was ordered having a reactive thiol group on its 3′ end (synthesis was done by Integrated DNA Technologies (IDT), Coralville, Iowa). The DNA was re-suspended in water and allowed to incubate at 37° for 30 minutes in the presence of 1,4 Dithio-DL-threitol (DTT, “Cleland reagent”) to break any disulfide bonds. The DNA was then passed through a PD 10 column (Amersham Pharmacia Biotech, Uppsala, Sweden) equilibrated in 1× PBS to (1) separate the thiol DNA away from DTT reagent, and (2) to bring the DNA up in a reaction buffer appropriate for GMBS conjugation. The DNA was evaporated to dryness. The amount of DNA probe was provided by the manufacturer. A 1.5 fold molar excess of DNA to protein was used. The anti-biotin mAb was also brought up in 1× PBS using PD10 column. The conjugating reagent was allowed to come to room temperature by allowing the reagent to sit at room temperature 30 minutes prior to use. The mAb was activated with 10×GMBS for 40 minutes and the reaction was quenched by adding 100 μL of 1M TRIS pH 7.2. The conjugate was confirmed by DNA band shift on a 2% agarose gel. The CY3 DNA capture probe was purified away from unlabeled DNA by use of a protein A/G agarose column (according to the manufacturer's (Pierce Chemical Company) standard protocol). The eluted fractions of this column were run on a 2% agarose column and confirmed the complete removal of the free DNA from the DNA antibody conjugate.

To demonstrate that the conjugate could localize a CY3 dendrimer, a microarray experiment was done. The conjugate was diluted 1:500 in 1× PBS binding buffer with 0.01% Tween20 and BSA (bovine serum albumin, a nonspecific blocking agent) and applied to a microarray of spotted biotin. The presence of spotted biotin was previously confirmed by fluorescently labeling streptavidin and staining. The DNA antibody conjugate solution was washed away by one 2 minute wash in 1× PBS/0.05% Tween20 followed by two 1 minute 1× PBS washes. A non-DNA antibody conjugate matching slide was also prepared. The CY3 3DNA dendrimer (Genisphere Inc.) was applied in the appropriate amount to the slides. Upon washing and drying, the slides were scanned and compared to negative control slide. The CY3 DNA capture labeled anti-biotin antibody yielded a dramatic CY3 fluorescence over each element of the biotin array, while the negative slide was completely blank across the entire slide for CY3 fluorescence.

### Example 6

### DNA Dendrimers for Encapsulation of Polymer Catalysts

A DNA polymer dendrimer comprising a nucleic acid sequence that hybridizes to a target nucleic acid molecule is incubated in a Ziegler Natta metallocene catalyst known to generate a polymer reaction, such as titanium chloride. The driving force between the host-guest encapsulation can be covalent, electrostatic, steric confinement, Van der Waals, or hydrogen bonding to the interior of the dendrimer). The dendrimer metal catalyst complex is separated by standard chromatography. The complex is used to probe a microarray containing the reverse complimentary sequence of the nucleic acid sequence attached to the dendrimer. The dendrimer catalyst complex hybridizes to the microarray on the basis of DNA sequence specificity. The array is washed and exposed to a polymer generating solution. The polymer solution contains a fraction of fluorescent monomers whose incorporation into the nascent polymer structure is localized to those areas where the reverse compliment DNA is contained. This polymer catalyst encapsulated into a dendrimer represents a new material composition useful also to many other applications in the emerging field of nanotechnology. The use of dendrimers to localize a polymer synthesis reaction is valuable beyond analyte detection and may be used also for novel nano-structure compositions as well.

### Example 7

### Incompatibility of DNA with Positively Charged NH₂ Starburst PAMAM Dendrimers

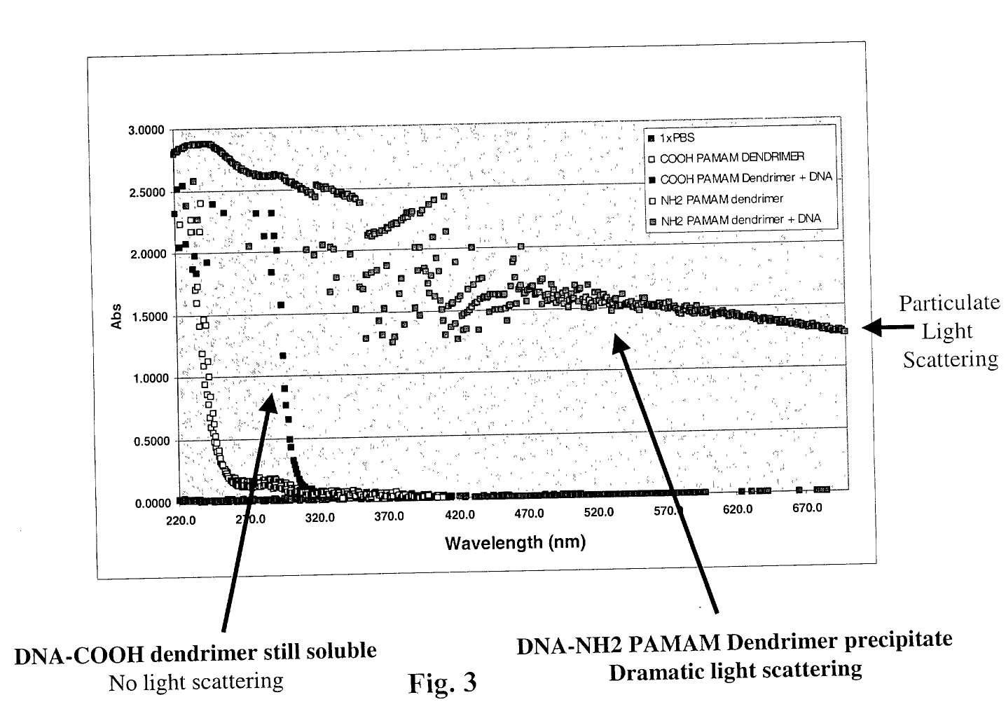

The admixture of negatively charged DNA with a positively charged high generation PAMAM dendrimer such as a NH₂ terminal functional groups results in an insoluble precipitate. Similarly, the admixture of a high generation (generation 5) positively charged dendrimer (NH₂ groups) with a negatively charged dendrimer (COOH groups) results in an insoluble precipitate. The addition of surfactants (e.g., SDS) or positively charged polymers, for example, polylysine, may help assist in reducing these effects but will not likely eliminate this problem in the manufacture of covalently attached DNAs to dendrimers. However, DNA added to negatively charged (COOH generation 6.5; Dendritech Inc., Midland, Mich.) PAMAM dendrimers does not result in any precipitation. One way this has been demonstrated is by scanning the ultraviolet absorbance range of the Starburst PAMAM dendrimer polymers with and without the addition of DNA. As can be seen in FIG. 3, both the COOH PAMAM dendrimer and the NH₂ terminal PAMAM dendrimer yielded fairly equivalent absorbance spectra. A large absorbance peak for both polymers was observed at around 220 nm. Upon addition of DNA to the NH₂ dendrimer (generation 7 or generation 5), a visible precipitate was observed, resulting in a large light scatter effect across the entire wavelength scan. It is reasonable to believe that the negative phosphate backbone of the DNA has been attracted to the positive charges in the high display dendrimer and the hydrophobic bases have been exposed into solution, thus lowering the solubility of the polymers. Conversely, addition of DNA to a negatively charged COOH dendrimer resulted in no precipitate. As shown in FIG. 3, the only spectral modification of the DNA-dendrimer mixture is an elevated absorbance at the 260 nm region indicative of the addition of DNA. For this reason, DNA conjugations with dendrimers is required to be between either like charged molecules or wherein at least one molecule has a neutral charge. Therefore, the use of negatively charged dendrimers may be the preferred choice of terminal functional groups when using amplifying stains on DNA microarrays that are free of non-specific polymer-DNA interactions.

### Example 8

### Preparation of DNA (3′-NH₂, 5′ Cy5 dye) covalently attached to a Generation 6.5 COOH PAMAM dendrimer (512 COOH groups) by EDC chemistry.

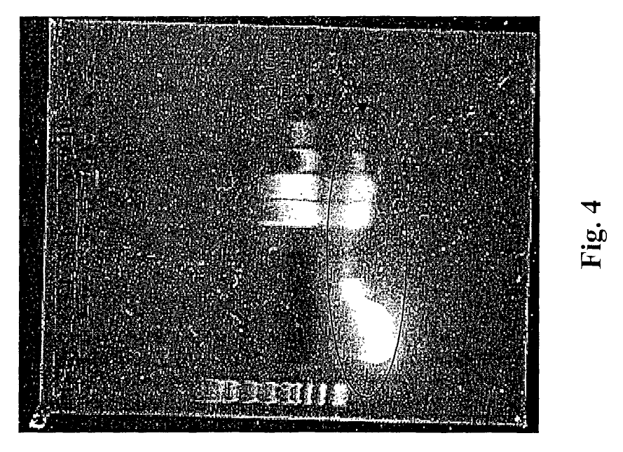

Molecules can be covalently attached to dendrimers by a number of coupling strategies. In the case of COOH terminal PAMAM dendrimers, amine containing DNAs, PNAs, RNAs, haptens, proteins or peptides can be coupled to the dendrimer by use of 1-Ethyl-3-(3-Dimethylaminopropyl) carboiimide hydrochloride (EDC) (Pierce Chemical Company). FIG. 4 depicts a scanned image of a 1.8% TBE Agarose gel showing the resulting conjugation of DNA oligonucleotides to a PAMAM dendrimer through the use of EDC, which can be used to couple an unreacted terminal COOH group on the dendrimer to an amine, for example, an amine in a dye, or on another molecule. DNA with either Cy3 or Cy5 dye can be attached to an unlabeled COOH PAMAM dendrimer. Adding labeled DNA to the COOH dendrimer without EDC coupling causes no gel shift in the DNA (lanes 4 and 5). The EDC coupled DNA dendrimer conjugates demonstrate a dramatic DNA band shift to a higher molecular weight species (lanes 6-10). The dye labeled oligonucleotides provided a measure for the position of the unattached DNAs.

### Example 9

### Comparison of DNA-Dye Conjugated to a Generation 6.5 COOH PAMAM and a Single Oligonucleotide Dye

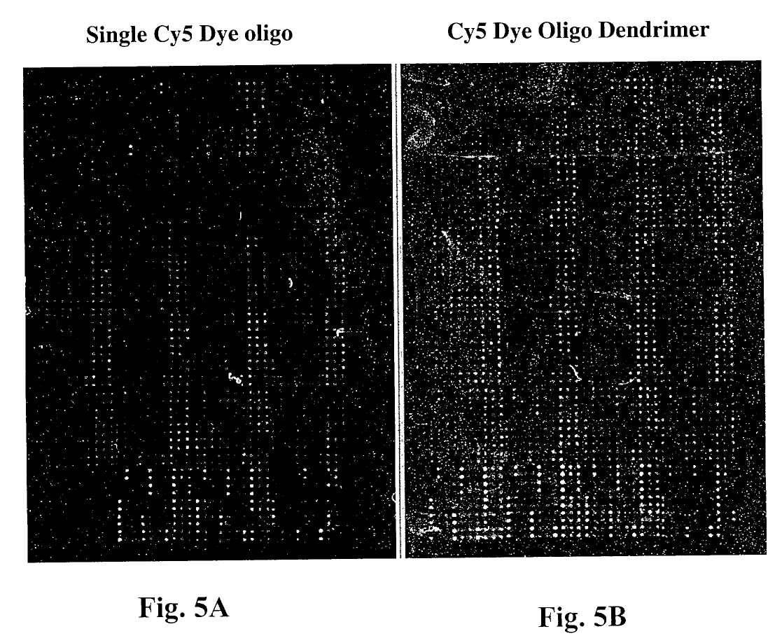

The amplification effect of staining a DNA slide with dye-labeled DNA dendrimers compared to staining with a single dye oligonucleotide is shown in FIGS. 5A and 5B. A slide was prepared with PCR amplicons relevant to a phenotypic screen. The Cy5 channel represents PCR amplicons for the IL1RN gene. FIG. 5A shows a scanned image of the fluorescence intensity for an oligo stain having just one Cy5 dye, while FIG. 5B is a scanned image of a slide stained with Cy5 labeled DNA PAMAM dendrimer. In each case, a bipartite probe, as described herein, was used to bridge the capture dye labeled DNA or dendrimer with the target DNA. After correction for background effects, the Cy5 labeled DNA PAMAM dendrimer slide was on average 4.5 times brighter than the oligo stain having just one Cy5 dye. This increase is without use of any follow-up dye-labeling of the dendrimer, use of dye-DNA decorators on the DNA dendrimer or even nested dye-DNA dendrimers (whose DNA sequence is the reverse compliment of the first DNA dendrimer) as described herein.

### Example 10

### Improved Biotin Detection on a Biotin Spotted Array Using a Biotin PAMAM Dendrimer with Streptavidin-Phycoerythrin Detection

Affymetrix GeneChips® hybridize fragmented biotinylated cRNA into an oligonucleotide array. The current protocol for staining an Affymetrix chip involves; (1) a stringency wash at 50° C.; (2) staining the slide with a streptavidin-phycoerythrin (SA-PE) complex; (3) staining the slide with a anti-streptavidin antibody that is biotinylated; and finally (4) staining again with streptavidin phycoerythrin complex. A biotinylated dendrimer was constructed in an attempt to improve the detection limits of the current SA-PE based stain. The biotin dendrimer was used as a replacement for the biotinylated anti-streptavidin stain (Stain 3 from the procedure given above). The COOH PAMAM dendrimer was equilibrated into 0.1M MES buffer (pH 5.5) using a PD10 column. The COOH dendrimer was combined with 2 mgs of EZ-Link™ Biotin-PEO-LC-Amine (Pierce Catalog Number 21347). A solution of EDC was prepared by adding 100 mgs of EDC to 1 ml of 0.1 M MES, pH 5.5 reaction buffer. A 100 μL volume of the EDC reaction was added to the COOH dendrimer solution and allowed to react overnight at 37° C. The biotin conjugated dendrimer mixture was then passed through a PD10 column equilibrated in 0.1 M PBS buffer. The 4th and 5th ml fractions were observed to contain dendrimer by absorbance. They were concentrated down to 0.2 ml by Millipore microcon YM-30 spin columns. This solution was then placed into a spectra/Por membrane (MWCO=12-14,000) and dialyzed for 2 days with 10 changes of distilled deionized water.

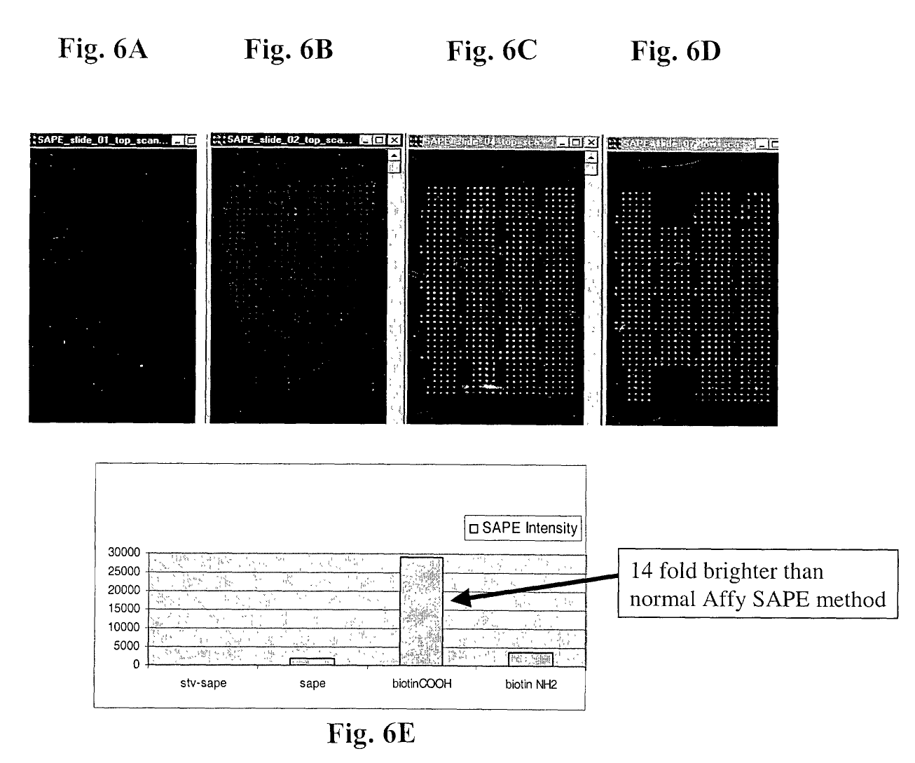

To first test the effect of replacing the biotin-anti-streptavidin stain with a biotinylated dendrimer, a series of test experiments were done on biotin spotted arrays. The biotin spotted arrays were constructed by spotting a solution of EZ-Link™ Biotin-PEO-LC-Amine (Pierce Catalog Number 21347) with BS³ chemical coupling agent onto aminosilane slides from Telechem International Corporation (Sunnyvale, Calif.). A Genetix Q-array equipped with Telechem pins was used to print the array. A 384 well plate was spotted in duplicate. FIGS. 6A-6D, depicts several biotin spotted slides that were stained in various ways yet all scanned by the same PMT and gain settings on a Packard 5000 microarray scanner. FIG. 6A is a scanned image of a stained slide shows the effect of using streptavidin to block the spotted biotin on the array prior to standard Affymetrix staining protocol (as described above). As expected, essentially all biotin sites were found blocked from SA-PE recognition. FIG. 6B is a scanned image of a slide showing SA-PE intensities for the normal Affymetrix stain procedure as described above. FIG. 6C is a scanned image of a slide showing the effect of replacing the biotinylated anti-streptavidin antibody stain (stain 3 above) with a biotin dendrimer made from a COOH PAMAM dendrimer (generation 6.5). This stain gave the highest signal amplification measured. FIG. 6D is a scanned image of a slide showing the effect of replacing the biotinylated anti-streptavidin antibody stain (stain 3 above) with a biotin dendrimer made from a NH₂ PAMAM dendrimer (generation 7). FIG. 6E shows the average intensity for all biotin spots on the microarray (average was approximately 710 spots). The biotin COOH PAMAM dendrimer gave a 14 fold increase in signal over the conventional Affymetrix stain.

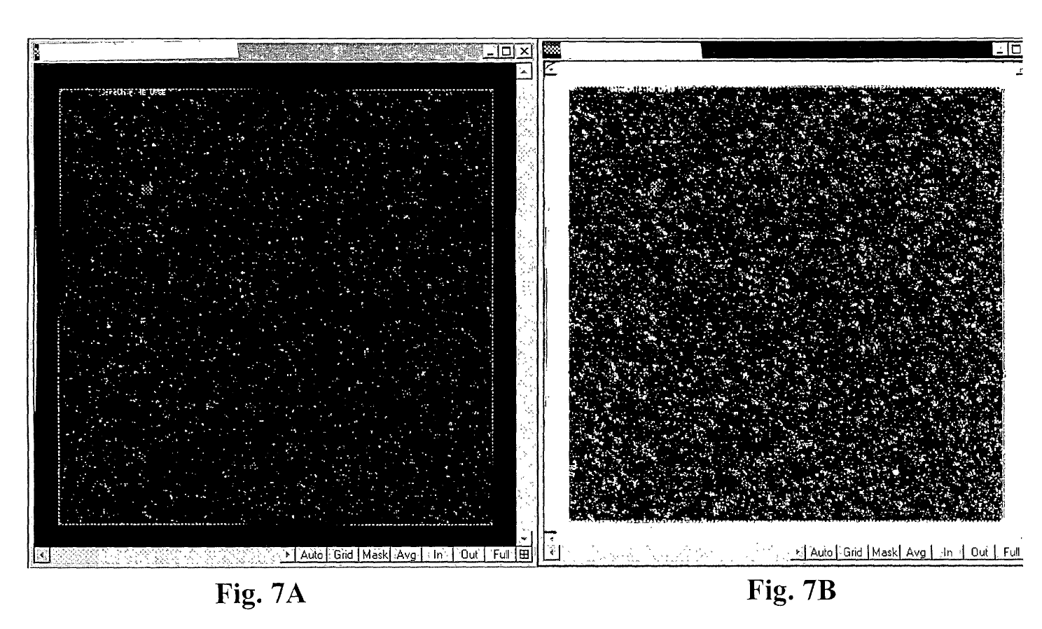

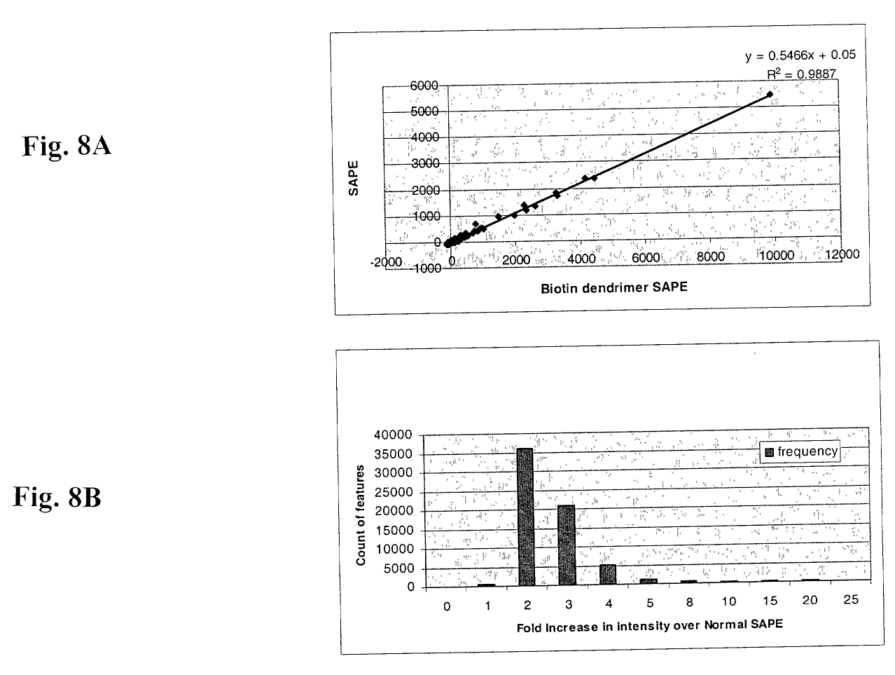

The biotinylated COOH PAMAM dendrimer signal amplifying modified stain was then employed on an Affymetrix chip. The Affymetrix U95B chip on the left in FIG. 7A shows the conventional SA-PE stain intensity for a 1 μg cRNA. FIG. 7B shows the intensity for a 1 μg of cRNA hybridized to the chip and stained using biotin COOH PAMAM dendrimer stain as a replacement for the biotinylated anti-streptavidin antibody stain (stain 3 above). FIG. 8A demonstrates the almost perfect correlation between the conventional SA-PE stain and the biotin dendrimer amplified stain. The high backgrounds on the outside of the array were reducible by using lower concentration of the biotin COOH PAMAM dendrimer. FIG. 8B is the histogram distribution (for roughly the first 300,000 features on each array) of the fold signal amplification yielded by using the biotin COOH PAMAM dendrimer stain. On average, the biotin dendrimer yielded a 2-3 fold increase in signal over conventional SA-PE based Affymetrix stain.

### Example 11

### Anti-biotin DNA Antibody Conjugate Capture of a Dendrimer (Immuno-dendrimer Stain)

As an example of immuno-dendrimer capture, as described herein, a DNA antibody conjugate was made (FIG. 9A). A DNA sequence (as given by Genisphere for a Cy5 labeled 3DNA dendrimer) was made by IDT which contained a Cy5 dye label at the 5′ end and a thiol group at the 3′ end. The DNA was conjugated to an anti-biotin mouse monoclonal antibody (IgG fraction from Jackson Immuno-research Labs) by adding 13 μL of 13.3 mM GMBS (coupling agent from Pierce Chemical Company) to a combined solution volume of 0.5 ml containing both 68 nmol (DNA) and 2.6 mgs protein in a 1×PBS buffer. The conjugation reaction was allowed to proceed overnight at 4° C. The reaction was quenched by adding 20 μL of 1M TRIS (pH 7.5). The DNA-antibody conjugate was then purified away from free DNA capture sequence by purification from an Immunopure® IgG affinityPak™ column (Pierce Chemical Company). The eluted fractions were shown to be pure DNA-anti-biotin MAb. The pure conjugate was equilibrated into phosphate buffer by passage through a PD-10 column. The phosphate buffer equilibrated DNA-antibody conjugate was then concentrated into a 0.3 ml volume by Millipore microcon spin concentrator columns (YM-30 columns). The conjugate was diluted in a 1:100 1× PBS solution and applied to a biotin spotted array (test slide). As a negative control, 1×PBS was applied to another matching biotin slide. After incubation of the slides at room temperature the positive control and negative control slides were washed in 1×PBS/0.01% Tween 20 buffer followed by a 1×PBS buffer wash. A Cy5 3DNA dendrimer solution (prepared as described by Genisphere Inc.) was applied to the slides. The dendrimer was allowed to bind for 1 hour at room temperature. The slides were then washed and scanned on a GSI Lumonics 5000 microarray scanner. Specific binding of the large 3DNA Genisphere DNA dendrimer by the DNA-antibody was observed in the test slide (FIGS. 9B), in contrast to the control slide, where specific binding was not observed (FIG. 9C). This immunodendrimer capture demonstrates capture of a large 12 million dalton dendrimer (Genisphere dendrimer) by an antibody capture agent. The COOH PAMAM Starburst dendrimers described herein are 116 kDa in mass. If a 6.5 generation DNA PAMAM dendrimer were fully conjugated by all 512 sites with a 30 mer DNA sequence one would expect a final mass of roughly 5.2 million daltons or half the size of the dendrimer that was used in this Example.

### Example 12

### Streptavidin Biotin “Bridge Capture” of a Dendrimer

An example of hapten capture of a fluorescent labeled DNA dendrimer is shown in FIG. 10A. A biotin spotted microarray was first stained with streptavidin and then washed. The streptavidin has four binding sites and when attached to biotins on the glass surface, streptavidin will be able to recognize additional biotins. A second stain was used with a DNA capture oligonucleotide containing a 5′ biotin label. The biotin oligonucleotide bound to the array (test slide). As a negative control a 1× PBS stain was used. The third and final stain was a Cy5 labeled DNA dendrimer from Genisphere (3DNA stain) containing a site that hybridizes to the capture oligo. The 3DNA stain was applied exactly as described by Genisphere. The dendrimer stain was allowed to bind for 1 hour. After the third stain, the slides were then washed and scanned on a GSI Lumonics 5000 microarray scanner. As shown in FIG. 10B, the test slide demonstrated a dramatic retention of the 3DNA dendrimer, compared to the negative control slide (FIG. 10C). It is reasonable to believe that a similar retention of a DNA-PAMAM dendrimer is possible.

While this invention has been particularly shown and described with references to preferred embodiments thereof, it will be understood by those skilled in the art that various changes in form and details may be made therein without departing from the scope of the invention encompassed by the appended claims.

What is claimed is:

1. A dendrimer comprising a nucleic acid molecule conjugated to a dendrimeric polymer, wherein said dendrimeric polymer is not a DNA dendrimeric polymer.

2. A method of detecting a target nucleic acid molecule, said method comprising the steps of:
a) contacting a target nucleic acid molecule with a bipartite capture probe comprising a target nucleic acid molecule capture region and a dendrimer capture region, under conditions that allow hybridization of said bipartite capture probe to said target nucleic acid molecule, thereby forming a target nucleic acid molecule bound capture probe;
b) contacting said target nucleic acid molecule bound capture probe with a detectably labeled dendrimer, said dendrimer comprising a dendrimeric polymer having one or more single stranded capture probe nucleic acid binding sequences attached to it, under conditions that allow hybridization of said dendrimer capture region of said bipartite capture probe to said single stranded capture probe binding sequence, thereby forming a labeled target nucleic acid molecule bound capture probe-dendrimer complex; and
c) detecting said labeled target nucleic acid molecule bound capture probe-dendrimer complex,
thereby detecting said target nucleic acid molecule.

3. A method of detecting a target nucleic acid molecule, said method comprising the steps of:
a) contacting a biotin-labeled target nucleic acid molecule with streptavidin under conditions that allow binding of said streptavidin to said biotin-labeled target nucleic acid molecule, thereby forming a biotin-labeled target nucleic acid molecule-streptavidin complex;
b) contacting said biotin-labeled target nucleic acid molecule-streptavidin complex with a biotin-labeled nucleic acid capture probe comprising a dendrimer capture region, under conditions that allow binding of said biotin-labeled capture probe to said biotin-labeled target nucleic acid molecule-streptavidin complex, thereby forming a biotin-labeled target nucleic acid bound capture probe;
c) contacting said biotin-labeled target nucleic acid bound capture probe with a detectably labeled dendrimer, said dendrimer comprising one or more single stranded capture probe nucleic acid binding sequences attached to it, under conditions that allow hybridization of said dendrimer capture region of said biotin-labeled target nucleic acid bound capture probe to said single stranded capture probe nucleic acid binding sequence, thereby forming a biotin-labeled target nucleic acid bound capture probe-detectably labeled dendrimer complex; and
d) detecting said biotin-labeled target nucleic acid bound capture probe-detectably labeled dendrimer complex,
thereby detecting said target nucleic acid molecule.

4. A method of detecting a target nucleic acid molecule, said method comprising the steps of:
a) contacting a hapten-labeled target nucleic acid molecule with a capture probe comprising a dendrimer capture region, said capture probe conjugated to an antibody that binds said hapten, under conditions that allow binding of said capture probe to said hapten-labeled target nucleic acid molecule, thereby forming a target nucleic acid bound capture probe;
b) contacting said hapten-labeled target nucleic acid bound capture probe with a detectably labeled dendrimer, said dendrimer comprising one or more single stranded capture probe nucleic acid binding sequences attached to it, under conditions that allow hybridization of said dendrimer capture region of said hapten-labeled target nucleic acid bound capture probe to said single stranded capture probe nucleic acid binding sequence, thereby forming a hapten-labeled target nucleic acid bound capture probe-detectably labeled dendrimer complex; and
c) detecting said hapten-labeled target nucleic acid bound capture probe-detectably labeled dendrimer complex,
thereby detecting said target nucleic acid molecule.

5. A method of detecting a target nucleic acid molecule, said method comprising the steps of:
a) contacting a biotin-labeled target nucleic acid molecule and a biotin-labeled dendrimer with a streptavidin molecule under conditions that allow binding of said biotin-labeled target nucleic acid molecule and said biotin-labeled dendrimer to same said streptavidin molecule, thereby forming a biotin-labeled target nucleic acid molecule-dendrimer complex; and
b) detecting said biotin-labeled target nucleic acid molecule-dendrimer complex,
thereby detecting said target nucleic acid molecule.

6. A method of detecting a target nucleic acid molecule, said method comprising the steps of:
a) contacting a biotin-labeled target nucleic acid molecule with streptavidin under conditions that allow binding of said streptavidin to said biotin-labeled target nucleic acid molecule, thereby forming a target nucleic acid molecule-streptavidin complex;
b) contacting said target nucleic acid molecule-streptavidin complex with a biotin-labeled nucleic acid capture probe comprising a first dendrimer capture region, under conditions that allow binding of said biotin-labeled nucleic acid capture probe to said target nucleic acid molecule-streptavidin complex, thereby forming a target nucleic acid molecule bound capture probe;
c) contacting said target nucleic acid molecule bound capture probe with a first detectably labeled dendrimer, said first dendrimer comprising one or more single stranded capture probe nucleic acid binding sequences and one or more single stranded second biotin-labeled dendrimer capture nucleic acid sequences, under conditions that allow hybridization of said first capture region of said target nucleic acid bound capture probe to said single stranded capture probe nucleic acid binding sequence, thereby forming a target nucleic acid molecule bound capture probe-first dendrimer complex;
d) contacting said target nucleic acid molecule bound capture probe-first dendrimer complex with a second detectably labeled dendrimer comprising one or more nucleic acid sequences that hybridize to the first dendrimer, under conditions that allow hybridization of said target nucleic acid bound capture probe-first dendrimer complex to said second detectably labeled dendrimer, thereby forming a target nucleic acid molecule bound capture probe-nested detectably labeled dendrimer complex; and
e) detecting said target nucleic acid molecule bound capture probe-nested detectably labeled dendrimer complex,
thereby detecting said target nucleic acid molecule.

7. A method of detecting a target nucleic acid molecule, said method comprising the steps of:
a) contacting a hapten labeled target nucleic acid with a capture probe comprising a dendrimer capture region, said capture probe conjugated to an antibody that binds said hapten, under conditions that allow binding of said capture probe to said hapten-labeled target nucleic acid molecule, thereby forming a target nucleic acid bound capture probe;
b) contacting said hapten-labeled target nucleic acid bound capture probe with a dendrimer, said dendrimer comprising one or more nucleic acid sequences, said nucleic acid sequences further comprising single stranded capture probe nucleic acid binding sequences attached to it, under conditions that allow hybridization of said dendrimer capture region of said hapten-labeled target nucleic acid bound capture probe to said single stranded capture probe nucleic acid binding sequence, thereby forming a hapten-labeled target nucleic acid bound capture probe-dendrimer complex; and
c) detecting said hapten-labeled target nucleic acid bound capture probe-dendrimer complex by amplifying a nucleic acid sequence on the dendrimer, and detecting said amplification products,
thereby detecting said target nucleic acid molecule.
